(19) United States
(12) Patent Application Publication
(10) Pub. No.: US 2006/0089363 A1
(43) Pub. Date: Apr. 27, 2006

(54) Antibacterial agents

(75) Inventors: Andrew P. Ayscough, Cambridge (GB); Kenneth Keavey, Wokingham (GB)

Correspondence Address: BANNER & WITCOFF, 1001 G STREET N W, SUITE 1100, WASHINGTON, DC, 20001, US

(73) Assignee: VERNALIS (OXFORD) LIMITED, Wokingham (GB)

(21) Appl. No.: 10/510,600

(22) PCT Filed: Apr. 9, 2003
(86) PCT No.: PCT/GB03/01541

§ 371 Date: Oct. 24, 2005

(30) Foreign Application Priority Data
Apr. 13, 2002 (GB) 0208579.3

(51) Int. Cl. A61K 31/496; A61K 31/495; C07D 403/02; C07D 401/02; A61K 31/4545; A61K 31/445

(52) U.S. Cl. 514/253.01; 514/255.01; 514/317; 514/318; 544/360; 544/382; 546/194; 546/223

(57) ABSTRACT

Compounds of formula (II) have antibacterial activity: wherein Q represents a radical of formula —N(OH)CH(═0) or formula —C(═O)NH(OH); R₁ represents hydrogen, methyl or trifluoromethyl, or, except when Z is a radical of formula —N(OH)CH(═0), a hydroxy, halo or amino group; R₂ represents a group R₁₀-(V)ₙ-(ALK)ₘ- wherein R₁₀ represents hydrogen, or an optionally substituted C₁-C₆ alkyl, C₂-C₆ alkenyl, C₂-C₆ alkynyl, cycloalkyl, aryl, or heterocyclyl group, ALK represents a straight or branched divalent C₁-C₆ alkylene, C₂-C₆ alkenylene, or C2-C6 alkynylene radical, and may be interrupted by one or more non-adjacent —NH—, —O— or —S— linkages, V represents —NH—, —O— or —S—, m and n are independently 0 or 1; R₃ represents the side chain of a natural or non-natural alpha amino acid; R₄ represents hydrogen or C₁-C₃ alkyl; Y represents N or CH; ring A is optionally substituted on one or more ring carbon atoms by C₁-C₃ alkyl, C₁-C₃ alkoxy, or halo; and R₅ represents a group (IIA): wherein m is 0 or 1; Alk¹ represents a divalent C₁-C₃ alkylene radical; Z represents hydrogen or an optionally substituted cycloalkyl, phenyl or heterocyclic group.

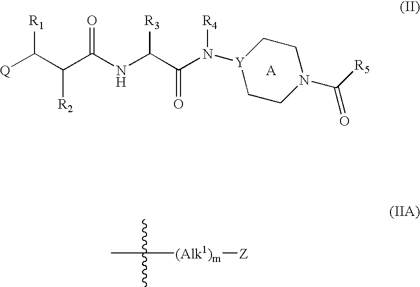

This invention relates to novel hydroxamic acid and N-formyl hydroxylamine derivatives having antibacterial activity, to methods of treatment using such compounds; and to pharmaceutical and veterinary compositions comprising such compounds.

### BACKGROUND OF THE INVENTION

Many classes of antibacterial agents are known, including the penicillins and cephalosporins, tetracyclines, sulfonamides, monobactams, fluoroquinolones and quinolones, aminoglycosides, glycopeptides, macrolides, polymyxins, lincosamides, trimethoprim and chloramphenicol. The fundamental mechanisms of action of these antibacterial classes vary.

Bacterial resistance to many known antibacterials is a growing problem. Accordingly there is a continuing need in the art for alternative antibacterial agents, especially those which have mechanisms of action fundamentally different from the known classes.

Amongst the Gram-positive pathogens, such as Staphylococci, Streptococci, Mycobacteria and Enterococci, resistant strains have evolved/arisen which makes them particularly difficult to eradicate. Examples of such strains are methicillin resistant Staphylococcus aureus (MRSA), methicillin resistant coagulase negative Staphylococci (MRCNS), penicillin resistant Streptococcus pneumoniae and multiply resistant Enterococcus faecium.

Pathogenic bacteria are often resistant to the aminoglycoside, β-lactam (penicillins and cephalosporins), and chloramphenicol types of antibiotic. This resistance involves the enzymatic inactivation of the antibiotic by hydrolysis or by formation of inactive derivatives. The β-lactam (penicillin and cephalosporin) family of antibiotics are characterised by the presence of a β-lactam ring structure. Resistance to this family of antibiotics in clinical isolates is most commonly due to the production of a “penicillinase” (β-lactamase) enzyme by the resistant bacterium which hydrolyses the β-lactam ring thus eliminating its antibacterial activity.

Recently there has been an emergence of vancomycin-resistant strains of enterococci (Woodford N. 1998 Glycopeptide-resistant enterococci: a decade of experience. Journal of Medical Microbiology. 47(10):849-62). Vancomycin-resistant enterococci are particularly hazardous in that they are frequent causes of hospital based infections and are inherently resistant to most antibiotics. Vancomycin works by binding to the terminal D-Ala-D-Ala residues of the cell wall peptidioglycan precursor. The high-level resistance to vancomycin is known as VanA and is conferred by a genes located on a transposable element which alter the terminal residues to D-Ala-D-lac thus reducing the affinity for vancomycin.

In view of the rapid emergence of multidrug-resistant bacteria, the development of antibacterial agents with novel modes of action that are effective against the growing number of resistant bacteria, particularly the vancomycin resistant enterococci and β-lactam antibiotic-resistant bacteria, such as methicillin-resistant Staphylococcus aureus, is of utmost importance.

### BRIEF DESCRIPTION OF THE INVENTION

This invention is based on the finding that certain hydroxamic acid and N-formyl hydroxylamine derivatives have antibacterial activity, and makes available a new group of antibacterial agents. It has been found that the compounds with which this invention is concerned are antibacterial with respect to a range of bacteria, with potency against Gram-positive organisms generally being greater than against Gram-negatives. Many of the compounds of the invention show activity against bacteria responsible for respiratory infections, such as Streptococcus pneumoniae and Haemophilus influenzae.

Although it may be of interest to establish the mechanism of action of the compounds with which the invention is concerned, it is their ability to inhibit bacterial growth that makes them useful. However, it is presently believed that their antibacterial activity is due, at least in part, to intracellular inhibition of bacterial polypeptide deformylase (PDF; EC 3.5.1.31).

All ribosome-mediated synthesis of proteins starts with a methionine residue. In prokaryotes the methionyl moiety carried by the initiator tRNA is N-formylated prior to its incorporation into a polypeptide. Consequently, N-formylmethionine is always present at the N-terminus of a nascent bacterial polypeptide. However, most mature proteins do not retain the N-formyl group or the terminal methionine residue. Deformylation is required prior to methionine removal, since methionine aminopeptidase does not recognise peptides with an N-terminal formylmethionine residue (Solbiati et al., J. Mol. Biol. 290:607-614, 1999). Deformylation is, therefore, a crucial step in bacterial protein biosynthesis and the enzyme responsible, PDF, is essential for normal bacterial growth. Although the gene encoding PDF (det) is present in all pathogenic bacteria for which sequences are known (Meinnel et al., J. Mol. Biol, 266.939-49, 1997), it has no eukaryotic counterpart, making it an attractive target for antibacterial chemotherapy.

The isolation and characterisation of PDF has been facilitated by an understanding of the importance of the metal ion in the active site (Groche et al., Biophys. Biochem. Res. Commun., 246:324-6,1998). The Fe²⁺ form is highly active in vivo but is unstable when isolated due to oxidative degradation (Rajagopalan et al., J. Biol. Chem. 273:22305-10, 1998). The Ni²⁺ form of the enzyme has specific activity comparable with the ferrous enzyme but is oxygen-insensitive (Ragusa et al., J. Mol. Biol. 1998, 280:515-23, 1998). The Zn²⁺ enzyme is also stable but is almost devoid of catalytic activity (Rajagopalan et al., J. Am. Chem. Soc. 119:12418-12419, 1997).

Several X-ray crystal structures and NMR structures of E. coli PDF, with or without bound inhibitors, have been published (Chan et al., Biochemistry 36:13904-9, 1997; Becker et al., Nature Struct. Biol. 5:1053-8, 1998; Becker et al., J. Biol. Chem. 273:11413-6, 1998; Hao et al., Biochemistry, 38:4712-9, 1999; Dardel et al., J. Mol. Biol. 280:501-13, 1998; O'Connell et al., J. Biomol. NMR, 13:311-24, 1999), indicating similarities in active site geometry to metalloproteinases such as thermolysin and the metzincins.

Recently the substrate specificity of PDF has been extensively studied (Ragusa et al., J. Mol. Biol. 289:1445-57, 1999; Hu et al., Biochemistry 38:643-50, 1999; Meinnel et al., Biochemistry, 38:4287-95, 1999). These authors conclude that an unbranched hydrophobic chain is preferred at P1′, while a wide variety of P2′ substituents are acceptable and an aromatic substituent may be advantageous at the P3′ position. There have also been reports that small peptidic compounds containing an H-phosphonate (Hu et al., Bioorg. Med. Chem. Left., 8:2479-82, 1998) or thiol (Meinnel et al., Biochemistry, 38:4287-95, 1999) metal binding group are micromolar inhibitors of PDF. Peptide aldehydes such as calpeptin (N-Cbz-Leu-norleucinal) have also been shown to inhibit PDF (Durand et al., Arch. Biochem. Biophys., 367:297-302, 1999). However, the identity of the metal binding group and its spacing from the rest of the molecule (“recognition fragment”) has not been studied extensively. Furthermore, non-peptidic PDF inhibitors, which may be desirable from the point of view of bacterial cell wall permeability or oral bioavailability in the host species, have not been identified.

### RELATED PRIOR ART

Certain N-formyl hydroxylamine derivatives have previously been claimed in the patent publications listed below, although very few examples of such compounds have been specifically made and described:

| | |
|---|---|
| EP-B-0236872 | (Roche) |
| WO 92/09563 | (Glycomed) |
| WO 92/04735 | (Syntex) |
| WO 95/19965 | (Glycomed) |
| WO 95/22966 | (Sanofi Winthrop) |
| WO 95/33709 | (Roche) |
| WO 96/23791 | (Syntex) |
| WO 96/16027 | (Syntex/Agouron) |
| WO 97/03783 | (British Biotech) |
| WO 97/18207 | (DuPont Merck) |
| WO 98/38179 | (GlaxoWellcome) |
| WO 98/47863 | (Labs Jaques Logeais) |

The pharmaceutical utility ascribed to the N-formyl hydroxylamine derivatives in those publications is the ability to inhibit matrix metalloproteinases (MMPs) and in some cases release of tumour necrosis factor (TNF), and hence the treatment of diseases or conditions mediated by those enzymes, such as cancer and rheumatoid arthritis.

In addition to these, U.S. Pat. No. 4,738,803 (Roques et al.) also discloses N-formyl hydroxylamine derivatives, however, these compounds are disclosed as enkephalinase inhibitors and are proposed for use as antidepressants and hypotensive agents. Also, WO 97/38705 (Bristol-Myers Squibb) discloses certain N-formyl hydroxylamine derivatives as enkephalinase and angiotensin converting enzyme inhibitors.

Our copending International Patent Application No. WO 99/39704 describes and claims, inter alia, the use of a compound of formula (I) or a pharmaceutically or veterinarily acceptable salt thereof in the preparation of an antibacterial composition: 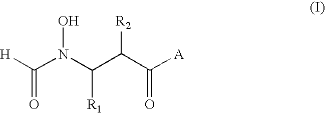
wherein R₁ represents hydrogen, C₁-C₈ alkyl or C₁-C₆ alkyl substituted by one or more halogen atoms; R₂ represents a substituted or unsubstituted C₁-C₆ alkyl, cycloalkyl(C₁-C₆ alkyl)- or aryl(C₁-C₆ alkyl)- group; and A represents a group of formula (IA), or (IB): 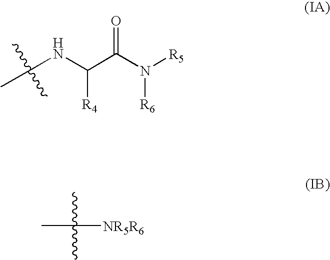
wherein R₄ represents the side chain of a natural or non-natural alpha amino acid, and R₅ and R₆ when taken together with the nitrogen atom to which they are attached form an optionally substituted saturated heterocyclic ring of 3 to 8 atoms which ring is optionally fused to a carbocyclic or second heterocyclic ring.

Very many hydroxamic acid derivatives are known. Many have been disclosed as having matrix metalloproteinase (MMP) inhibitory activity, and thus to be potentially useful for the treatment of diseases mediated by MMPS, for example cancer, arthritides, and conditions involving tissue remodeling such as wound healing, and restenosis. In addition our International Patent Application No. WO 99/59568 describes the use of analogues of the N-formylhydroxylamine derivatives of WO 99/39704 (wherein the N-formylhydroxylamine group is replaced by a hydroxamic acid group) in the preparation of an antibacterial composition.

### BRIEF DESCRIPTION OF THE INVENTION

This invention relates to a group of antibacterially active hydroxamic acid and and N-formyl hydroxylamine compounds which differ in structure from those of International Patent Applications Nos. WO 99159568 and WO 99/39704, principally in the nature of the —NR₅R₆ group (see formulae (I), (IA) above and the hydroxamic acid analogues thereof). In those applications, R₅ may be C₁-C₆ alkyl and R₆ may be an optionally substituted heterocuclic ring. The term “optionally substituted” as used in relation to the saturated heterocyclic ring R₆ is defined as meaning certain specific substituents. In the present compounds, the group R₅ is also C₁-C₆ alkyl and R₆ is also substituted heterocyclic ring, namely piperidinyl or piperazinyl, but the substituents are different from those permitted by WO 99/59568 and WO 99/39704. The group —NR₅R₆ of the N-formyl hydroxylamines and hydroxamic acids of the invention is also believed to distinguish the present compounds from those known in the MMP, TNF, ACE, and enkephalinase inhibitor art.

### DETAILED DESCRIPTION OF THE INVENTION

The present invention provides a compound of formula (II), or a pharmaceutically or veterinarily acceptable salt, hydrate or solvate thereof 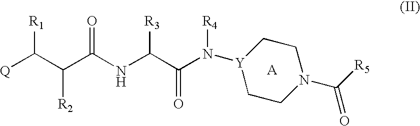
wherein

Q represents a radical of formula —N(OH)CH(═O) or formula —C(═O)NH(OH);

R₁ represents hydrogen, methyl or trifluoromethyl, or, except when Z is a radical of formula —N(OH)CH(═O), a hydroxy, halo or amino group;

R₂ represents a group R₁₀-(V)ₙ-(ALK)ₘ- wherein
R₁₀ represents hydrogen, or a C₁-C₆ alkyl, C₂-C₆ alkenyl, C₂-C₆ alkynyl, cycloalkyl, aryl, or heterocyclyl group, any of which may be unsubstituted or substituted by (C₁-C₆)alkyl, (C₁-C₆)alkoxy, hydroxy, mercapto, (C₁-C₆)alkylthio, amino, halo (including fluoro, chloro, bromo and iodo), trifluoromethyl, cyano, nitro, oxo, —COOH, —CONH₂, —COOR^{A}, —NHCOR^{A}, —CONHR^{A}, —NHR^{A}, —NR^{A}R^{B}, or —CONR^{A}R^{B} wherein R^{A} and R^{B} are independently a (C₁-C₆)alkyl group and ALK represents a straight or branched divalent C₁-C₆ alkylene, C₂-C₆ alkenylene, or C₂-C₆ alkynylene radical, and may be interrupted by one or more non-adjacent —NH—, —O— or —S— linkages,
V represents —NH—, —O— or —S—, and
m and n are independently 0 or 1;

R₃ represents the side chain of a natural or non-natural alpha amino acid;

R₄ represents hydrogen or C₁-C₃ alkyl;

Y represents N or CH;

ring A is optionally substituted on one or more ring carbon atoms by C₁-C₃ alkyl, C₁-C₃ alkoxy, or halo; and

R₅ represents a group (IIA), 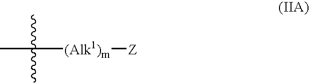
wherein

m is 0 or 1;

Alk¹ represents a divalent C₁-C₃ alkylene radical;

Z represents hydrogen or cycloalkyl, phenyl or heterocyclic which is optionally substituted by
(C₁-C₆)alkyl,
phenyl

monocyclic 5 or 6-membered heterocyclic,

benzyl,

phenoxy, or (C₁-C₆)alkoxy,

phenylthio or (C₁-C₆)alkylthio, any of which is in turn optionally substituted by:
hydroxy or mercapto,
trifluoromethyl,
oxo,
nitro,
cyano (—CN)
bromo, chloro, fluoro, or iodo
—COOH, or —COOR^{A},
—CONH₂, —CONHR^{A}, or —CONR^{A}R^{B}
—COR^{A}, —SO₂R^{A},
—NHCOR^{A},
—NH₂, —NHR^{A}, or —NR^{A}R^{B},
   wherein R^{A} and R^{B} are independently a (C₁-C₆) alkyl group, or R^{A} and R^{B} taken together with the nitrogen atom to which they are attached form a 5- or 6-membered heterocyclic ring which may be substituted by (C₁C₃)alkyl, hydroxy, or hydroxy(C₁-C₃)alkyl.

In a subset of the compounds of the invention, Z represents a cycloalkyl, phenyl or monocyclic heterocyclic ring, which is optionally substituted by
(C₁-C₆)alkyl, (C₂-C₆)alkenyl, or (C₂-C₆)alkynyl,
phenyl, or halophenyl,
trifluoromethyl,
monocyclic 5 or 6-membered hetrocyclic,
benzyl, or halophenylmethyl,
hydroxy, phenoxy, (C₁-C₆)alkoxy, or hydroxy(C₁-C₆)alkyl,
mercapto, (C₁-C₆)alkylthio or mercapto(C₁-C₆)alkyl,

oxo,

nitro,

cyano (—CN)

bromo, chloro, fluoro, or iodo

—COOH, or —COOR^{A},

—CONH₂, —CONHR^{A}, or—CONR^{A}R^{B}

—COR^{A}, —SO₂R^{A},

—NHCOR^{A},

—NH₂, —NHR^{A}, or —NR^{A}R^{B},
wherein R^{A} and R^{B} are independently a (C₁-C₆) alkyl group, or R^{A} and R^{B} taken together with the nitrogen atom to which they are attached form a 5- or 6-membered heterocyclic ring which may be substituted by (C₁C₃)alkyl, hydroxy, or hydroxy(C₁-C₃)alkyl.

In another aspect, the invention provides a method for the treatment of bacterial infections in humans and non-human mammals, which comprises administering to a subject suffering such infection an antibacterially effective dose of a compound of formula (II) as defined above.

In a further aspect of the invention there is provided a method for the treatment of bacterial contamination by applying an antibacterially effective amount of a compound of formula (II) as defined above to the site of contamination.

The compounds of formula (II) as defined above may be used as component(s) of antibacterial cleaning or disinfecting materials.

On the hypothesis that the compounds (II) act by inhibition of intracellular PDF, the most potent antibacterial effect may be achieved by using compounds which efficiently pass through the bacterial cell wall. Thus, compounds which are highly active as inhibitors of PDF in vitro and which penetrate bacterial cells are preferred for use in accordance with the invention. It is to be expected that the antibacterial potency of compounds which are potent inhibitors of the PDF enzyme in vitro, but are poorly cell penetrant, may be improved by their use in the form of a prodrug, ie a structurally modified analogue which is converted to the parent molecule of formula (II), for example by enzymic action, after it has passed through the bacterial cell wall.

As used herein the term “(C₁-C₆)alkyl” means a straight or branched chain alkyl moiety having from 1 to 6 carbon atoms, including for example, methyl, ethyl, n-propyl, isopropyl, n-butyl, isobutyl, sec-butyl, t-butyl, n-pentyl and n-hexyl.

As used herein the term “divalent (C₁-C₃)alkylene radical” means a saturated hydrocarbon chain having from 1 to 3 carbon atoms and two unsatisfied valencies.

As used herein the term “(C₂-C₆)alkenyl” means a straight or branched chain alkenyl moiety having from 2 to 6 carbon atoms having at least one double bond of either E or Z stereochemistry where applicable. The term includes, for example, vinyl, allyl, 1- and 2-butenyl and 2-methyl-2-propenyl.

As used herein the term “C₂-C₆ alkynyl” refers to straight chain or branched chain hydrocarbon groups having from two to six carbon atoms and having in addition one triple bond. This term would include for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein the term “cycloalkyl” means a saturated alicyclic moiety having from 3-8 carbon atoms and includes, for example, cyclopropyl, cyclobutyl, cyclopentyl, cyclohexyl, cycloheptyl and cyclooctyl.

As used herein the term “heteroaryl” refers to a 5- or 6-membered aromatic ring containing one or more heteroatoms. Illustrative of such groups are thienyl, furyl, pyrrolyl, imidazolyl, benzimidazolyl, thiazolyl, pyrazolyl, isoxazolyl, isothiazolyl, triazolyl, thiadiazolyl, oxadiazolyl, pyridinyl, pyridazinyl, pyrimidinyl, pyrazinyl, triazinyl.

As used herein the unqualified term “heterocyclyl” or “heterocyclic” includes “heteroaryl” as defined above, and in particular means a 5-7 membered aromatic or non-aromatic heterocyclic ring containing one or more heteroatoms selected from S, N and O, including for example, pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzofuranyl, pyranyl, isoxazolyl, benzimidazolyl, methylenedioxyphenyl, maleimido and succinimido groups.

As used herein the terms “side chain of a natural alpha-amino acid” and Aside chain of a non-natural alpha-amino acid≅mean the group R^{x} in respectively a natural and non-natural amino acid of formula NH₂—CH(R^{x})—COOH.

Examples of side chains of natural alpha amino acids include those of alanine, arginine, asparagine, aspartic acid, cysteine, cystine, glutamic acid, histidine, 5-hydroxylysine, 4-hydroxyproline, isoleucine, leucine, lysine, methionine, phenylalanine, proline, serine, threonine, tryptophan, tyrosine, valine, α-aminoadipic acid, α-amino-n-butyric acid, 3,4-dihydroxyphenylalanine, homoserine, α-methylserine, ornithine, pipecolic acid, and thyroxine.

In natural alpha-amino acid side chains which contain functional substituents, for example amino, carboxyl, hydroxy, mercapto, guanidyl, imidazolyl, or indolyl groups as in arginine, lysine, glutamic acid, aspartic acid, tryptophan, histidine, serine, threonine, tyrosine, and cysteine, such functional substituents may optionally be protected.

Likewise, in the side chains of non-natural alpha amino acids which contain functional substituents, for example amino, carboxyl, hydroxy, mercapto, guanidyl, imidazolyl, or indolyl groups, such functional substituents may optionally be protected.

The term “protected” when used in relation to a functional substituent in a side chain of a natural or non-natural alpha-amino acid means a derivative of such a substituent which is substantially non-functional. The widely used handbook by T. W. Greene and P. G. Wuts “Protective Groups in Organic Synthesis” Second Edition, Wiley, New York, 1991 reviews the subject. For example, carboxyl groups may be esterified (for example as a C₁-C₆ alkyl ester), amino groups may be converted to amides (for example as a NHCOC₁-C₆ alkyl amide) or carbamates (for example as an NHC(═O)OC₁-C₆ alkyl or NHC(═O)OCH₂Ph carbamate), hydroxyl groups may be converted to ethers (for example an OC₁-C₆ alkyl or a O(C₁-C₆ alkyl)phenyl ether) or esters (for example a OC(═O)C₁-C₆ alkyl ester) and thiol groups may be converted to thioethers (for example a tert-butyl or benzyl thioether) or thioesters (for example a SC(═O)C₁-C₆ alkyl thioester).

There are several actual or potential chiral centres in the compounds according to the invention because of the presence of asymmetric carbon atoms. The presence of several asymmetric carbon atoms gives rise to a number of diastereoisomers with R or S stereochemistry at each chiral centre. The invention includes all such diastereoisomers and mixtures thereof. Currently, the preferred stereoconfiguration of the carbon atom carrying the R₂ group is R; that of the carbon atom carrying the R₄ group (when asymmetric) is S; and that of the carbon atom carrying the R₁ group (when asymmetric) is R.

In the compounds of the invention:

When Q is a radical of formula —N(OH)CH(═O), R₁ may be, for example, hydrogen, methyl, or trifuoromethyl. Hydrogen is currently preferred.

When Q is a radical of formula —C(═O)NH(OH), R₁ may be, for example, hydrogen, methyl, trifluoromethyl, hydroxy, halo or amino. Again hydrogen is currently preferred.

R₂ may be, for example:
optionally substituted C₁-C₈ alkyl, C₃-C₆ alkenyl, C₃-C₆ alkynyl or cycloalkyl;
phenyl(C₁-C₆ alkyl)-, phenyl(C₃-C₆ alkenyl)- or phenyl(C₃-C₆ alkynyl)-optionally substituted in the phenyl ring;
cycloalkyl(C₁-C₆ alkyl)- for example cycloalkylmethyl, cycloalkyl(C₃-C₆ alkenyl)- or cycloalkyl(C₃-C₆ alkynyl)-, optionally substituted in the cycloalkyl ring;
heterocyclyl(C₁-C₆ alkyl)-, heterocyclyl(C₃-C₆ alkenyl)- or heterocyclyl(C₃-C₆ alkynyl)- optionally substituted in the heterocyclyl ring; or
(C₁-C₃)alkyl—S-(C₁-C₃)alkyl-, or (C₁-C₃)alkyl-O-(C₁-C₃)alkyl-.

Specific examples of R₂ groups include
methyl, ethyl, n- and iso-propyl, n- and iso-butyl, n-pentyl, iso-pentyl 3-methyl-but-1-yl, n-hexyl, n-heptyl, n-acetyl, n-octyl, methylsulfanylethyl, ethylsulfanylmethyl, 2-methoxyethyl, 2-ethoxyethyl, 2-ethoxymethyl, 3-hydroxypropyl, allyl, 3-phenylprop-3-en-1-yl, prop-2-yn-1-yl, 3-phenylprop-2-yn-1-yl, 3-(2-chlorophenyl)prop-2-yn-1-yl, but-2-yn-1-yl, cyclopentyl, cyclohexyl, cyclopentylmethyl, cyclopentylethyl, cyclopentylpropyl, cyclohexylmethyl, cyclohexylethyl, cyclohexylpropyl, furan-2-ylmethyl, furan-3-methyl, tetrahydrofuran-2-ylmethyl, tetrahydrofuran-2-ylmethyl, piperidinylmethyl, phenylpropyl, 4-chlorophenylpropyl, 4-methylphenylpropyl, 4-methoxyphenylpropyl, benzyl, 4-chlorobenzyl, 4-methylbenzyl, and 4-methoxybenzyl.
Presently preferred groups at R₂ are (C₁-C₆)alkyl-, cycloalkylmethyl-, (C₁-C₃)alkyl-S-(C₁-C₃)alkyl-, or (C₁-C₃)alkyl-O-(C₁-C₃)alkyl-, especially n-propyl, n-butyl, n-pentyl, cyclopentylmethyl, cyclopentylethyl, cyclohexylmethyl or cyclohexylethyl.

R₃ may be, for example
the characterising group of a natural α amino acid, for example benzyl, or 4-methoxyphenylmethyl, in which any functional group may be protected, any amino group may be acylated and any carboxyl group present may be amidated; or
a group -[Alk]ₙR₉ where Alk is a (C₁-C₆)alkylene or (C₂-C₆)alkenylene group optionally interrupted by one or more —O—, or —S— atoms or —N(R₁₂)— groups [where R₁₂ is a hydrogen atom or a (C₁-C₈)alkyl group], n is 0 or 1, and R₉ is hydrogen or an optionally substituted phenyl, aryl, heterocyclyl, cycloalkyl or cycloalkenyl group or (only when n is 1) R₉ may additionally be hydroxy, mercapto, (C₁-C₆)alkylthio, amino, halo, trifluoromethyl, nitro, —COOH, —CONH₂, —COOR^{A}, —NHCOR^{A}, —CONHR^{A}, —NHR^{A}, —NR^{A}R^{B}, or —CONR^{A}R^{B} wherein R^{A} and R^{B} are independently a (C₁-C₆)alkyl group; or
a benzyl group substituted in the phenyl ring by a group of formula —OCH₂COR₈ where R₈ is hydroxyl, amino, (C₁-C₆)alkoxy, phenyl(C₁-C₆)alkoxy, (C₁-C₆)alkylamino, di((C₁-C₆)alkyl)amino, phenyl(C₁-C₆)alkylamino; or
a heterocyclic(C₁-C₆)alkyl group, either being unsubstituted or mono- or di-substituted in the heterocyclic ring with halo, nitro, carboxy, (C₁-C₆)alkoxy, cyano, (C₁-C₆)alkanoyl, trifluoromethyl (C₁-C₆)alkyl, hydroxy, formyl, amino, (C₁-C₈)alkylamino, di-(C₁-C₆)alkylamino, mercapto, (C₁-C₆)alkylthio, hydroxy(C₁-C₆)alkyl, mercapto(C₁-C₆)alkyl or (C₁-C₆)alkylphenylmethyl; or

a group —CRₐR_{b}R_{c} in which:
each of Rₐ, R_{b} and R_{c} is independently hydrogen, (C₁-C₆)alkyl, (C₂-C₆)alkenyl, (C₂-C₆)alkynyl, phenyl(C₁-C₆)alkyl, (C₃-C₈)cycloalkyl; or
R_{c} is hydrogen and Rₐ and R_{b} are independently phenyl or heteroaryl such as pyridyl; or
R_{c} is hydrogen, (C₁-C₆)alkyl, (C₂-C₆)alkenyl, (C₂-C₆)alkynyl, phenyl(C₁-C₆)alkyl, or (C₃-C₈)cycloalkyl, and Rₐ and R_{b} together with the carbon atom to which they are attached form a 3 to 8 membered cycloalkyl or a 5- to 6-membered heterocyclic ring; or
Rₐ, R_{b} and R_{c} together with the carbon atom to which they are attached form a tricyclic ring (for example adamantyl); or
Rₐ and R_{b} are each independently (C₁-C₆)alkyl, (C₂-C₆)alkenyl, (C₂-C₆)alkynyl, phenyl(C₁-C₆)alkyl, or a group as defined for R_{c} below other than hydrogen, or Rₐ and R_{b} together with the carbon atom to which they are attached form a cycloalkyl or heterocyclic ring, and R_{c} is hydrogen, —OH, —SH, halogen, —CN, —CO₂H, (C₁-C₄)perfluoroalkyl, —CH₂OH, —CO₂(C₁-C₆)alkyl, —O(C₁-C₆)alkyl, —O(C₂-C₈)alkenyl, —S(C₁-C₆)alkyl, —SO(C₁-C₆)alkyl, —SO₂(C₁-C₆) alkyl, —S(C₂-C₆)alkenyl, —SO(C₂-C₆)alkenyl, —SO₂(C₂-C₆)alkenyl or a group -Q-W wherein Q represents a bond or —O—, —S—, —SO— or —SO₂— and W represents a phenyl, phenylalkyl, (C₃-C₈)cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CO₂H, —CO₂(C₁-C₆)alkyl, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl.

Examples of particular R₃ groups include methyl, ethyl, benzyl, 4-chlorobenzyl, 4-hydroxybenzyl, phenyl, cyclohexyl, cyclohexylmethyl, pyridin-3-ylmethyl, tert-butoxymethyl, naphthylmethyl, iso-butyl, sec-butyl, tert-butyl, 1-benzylthio-1-methylethyl, 1-methylthio-1-methylethyl, 1-mercapto-1-methylethyl, 1-methoxy-1-methylethyl, 1-hydroxy-1-methylethyl, 1-fluoro-1-methylethyl, hydroxymethyl, 2-hydroxethyl, 2-carboxyethyl, 2-methylcarbamoylethyl, 2-carbamoylethyl, and 4-aminobutyl. Presently preferred R₃ groups include tert-butyl, iso-butyl, benzyl, isopropyl and methyl.

R₄ may be, for example methyl or ethyl. Methyl is currently preferred.

When m is 1, Alk¹ may be, for example, —(CH₂)— or —(CH₂CH₂)—.

Z may be, for example, a phenyl, pyridyl, thienyl, furanyl, pyranyl, pyrolyl, diazolyl, triazolyl, thiazolyl, thiadiazolyl, oxazolyl, ozadiazolyl, indolyl, benzisozazolyl, benzthiazolyl or imidazothiazolyl ring, optionally substituted as specified. Examples of optional substituents include methyl, methoxy, ethoxy, methoxymethyl, ethylthio, chloro, bromo, hydroxy, nitro, phenyl, 2- or 4-nitrophenyl, dimethylamino, dimethylaminophenyl, methylsulphonyl, dimethylaminosulphonyl, 3-pyridyl or 2-pyrazin-2-yl.

In one subgroup of the compounds of the invention, Z is a cyclopentyl, cyclohexyl, phenyl, morpholinyl, pyrimidin-2-yl, 1,2,3-thiadiazol-5-yl, 1,4-thiazol-5-yl, benzofuran-2-yl, 2-or 3-furanyl, 2- or 3-thienyl, 2- or 3-pyranyl, 2-, 3- or 4-pyrrolyl, 3-, 4- or 5-pyazolyl, 3-, 4- or 5-isoxazolyl, or 2-, 3- or 4-pyridyl ring any of which may optionally be substituted by hydroxy, methoxy, ethoxy, mercapto, methylthio, ethylthio, methyl, ethyl, trfluoromethyl, fluoro, chloro, amino, methylamino, or dimethylamino.

Examples of specific compounds of the invention are those of the Examples herein. . In those Examples, where a compound of formula (II) above wherein Q is an N-formylhydroxylamine radical —N(OH)CH(═O) is disclosed, it is to be understood that the equivalent compound wherein Q is a hydroxamate radical —C(═O)NH(OH) is also a specific compound of the invention, and vice versa.

Compounds of the invention in which Q is an N-formylhydroxyamino group may be prepared by deprotecting an O-protected N-formyl-N-hydroxyamino compound of formula (III): 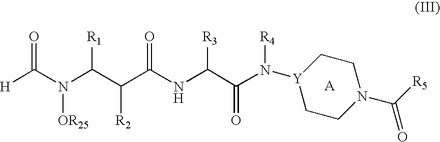
in which R₁, R₂, R₃, R₄, R₅ and Y are as defined in general formula (II) and R₂₅ is a hydroxy protecting group removable to leave a hydroxy group by hydrogenolysis or hydrolysis. Benzyl is a preferred R₂₅ group for removal by hydrogenolysis, and tert-butyl and tetrahydropyranyl are preferred groups for removal by acid hydrolysis.

Compounds of the invention in which Q is a hydroxamic acid group may be prepared by reacting the parent compound wherein Q is a carboxylic acid group (IIIA) 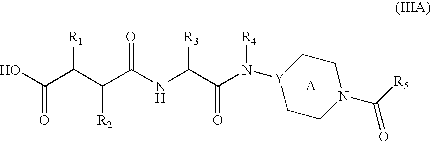
with hydroxylamine or an N- and/or O-protected hydroxylamine, and thereafter removing any O- or N-protecting groups

Compounds of formula (III) or (IIIA) may be prepared by causing an acid of formula (IV) or (IVA) or an activated derivative thereof to react with an amine of formula (V) 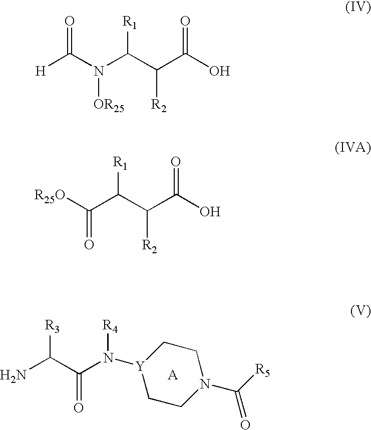
wherein R₁, R₂, R₃, R₄, R₅ and Y are as defined in general formula (II) except that any substituents in R₁ R₂, R₄, and R₅ which are potentially reactive in the coupling reaction may themselves be protected from such reaction, and R₂₅ is as defined in relation to formula (III) above, and optionally removing protecting groups R₁, R₂, R₄, and R₅.

Compounds of formula (V) may be prepared by N-acylation of a compound of formula (VI) with, for example, the acid chloride R₅COCl 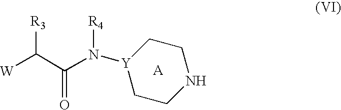
wherein R₄, R₅ and Y are as defined in general formula (V), and W is a protected amino group, and then removing the amino protecting group(s).

Compounds (II) of the invention may also be prepared by N-acylation of the corresponding piperazine or piperidine parent compound, as in the Examples herein.

Salts of the compounds of the invention include physiologically acceptable acid addition salts for example hydrochlorides, hydrobromides, sulphates, methane sulphonates, p-toluenesulphonates, phosphates, acetates, citrates, succinates, lactates, tartrates, fumarates and maleates. Salts may also be formed with bases, for example sodium, potassium, magnesium, and calcium salts.

Compositions with which the invention is concerned may be prepared for administration by any route consistent with the pharmacokinetic properties of the active ingredient(s).

Orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

Safe and effective dosages for different classes of patient and for different disease states will be determined by clinical trial as is required in the art. It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

The following Examples illustrate embodiments of the invention. In the Examples, the following abbreviations have been used throughout:
DCM Dichloromethane
DIEA Diisopropylethylamine
DMF Dimethylformamide
HATU O-(7-Azabenzotriazol-1-yl)-1,1,3,3-tetramethyluronium hexafluorophosphate
HOBt 1-Hydroxy-7-benzotriazole
HPLC High performance liquid chromatography
LRMS Low resolution mass spectrometry
NMR Nuclear Magnetic Resonance
PyAOP7 Azabenzotriazol-1-yl-oxy-tris-pyrrolidino-phosphonium hexafluorophosphate
rt Room temperature
RT Retention time
TBTU 2-(1H-Benzotriazole-1-yl)-1,1,3,3-tetramethyluronium tetrafluorophosphate
TFA Trifluoroacetic acid

¹H and ¹³C spectra were recorded using a Bruker DPX 250 spectrometer at 250.1 MHz (62.5 MHz for the ¹³C). Chemical shift values are expressed in 6 (ppm) and abbreviations are as follows: s=singlet, d=doublet, t=triplet, q=quartet, dd=double doublet, m=multiplet, b=broad and app=apparent. Mass spectra were obtained using a Perkin Elmer Sciex API 165. Analytical HPLC was run on a Beckman System Gold, using Waters Symmetry C18 column (50 mm, 4.6 mm) with 20 to 90% solvent B gradient (1.5 ml/min) as the mobile phase. [Solvent A: 0.05% TFA in 10% MeCN 90% water, Solvent B: 0.05% TFA in 10% water 90% MeCN, 5 min gradient time], detection wavelength at 220 or 214 nm. Preparative HPLC was run on a Gilson autoprep instrument using a C18 Waters delta pak (15 μm, 300 Å, 25 mm, 100 mm) with 10 to 90% solvent B gradient as the mobile phase at a flow rate of 15 ml/min. [Solvent A 10% MeCN/water; Solvent B: 10% water/MeCN, 8 min gradient time], UV detection was at 220 or 214 nm.

### Preparation of 2-[2-Cyclopentylmethyl-3-(formyl-hydroxy-amino)propionylamino]-3,3,N-trimethyl-N-piperidin-4-yl-butyramide (Intermediate 1)

Step 1

4-Methylamino-piperidine-1-carboxylic acid tert-butyl ester

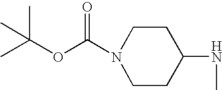

To a solution of tert-butoxy-piperidone (10 g, 50 mmol) in EtOH (100 ml) was added N-methylamine (10 ml of a 33% solution in EtOH) and palladium on carbon (1 g, 10% w/w), hydrogen was bubbled through the reaction mixture for 2 h. The mixture was stirred under a blanket of hydrogen for 18 h at RT. The Pd/C was filtered off and the solvent removed in vacuo to yield the title compound as a clear oil. LRMS: +ve ion 215 (M+1, 20%), 237 (M+Na, 15%). ¹H-NMR (250 MHz), δ (CDCl₃) 4.05 (2H, m), 2.80 (2H, m), 2.51 (1H, m), 2.45 (3H, s), 1.87 (2H, m), 1.62 (1H, s), 1.45 (9H, s), 1.25 (2H, m).

Step 2

4-[(2-Benzyloxycarbonylamino-3,3-dimethyl-butyryl-methyl-amino]-piperidine-1-carboxylic acid tert-butyl ester

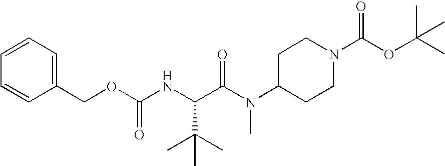

To a solution of Cbz-protected tert-leucine (500 mg, 1.9 mmol) in dichloromethane (10 ml) was added PyAOP (1.08 g, 2.1 mmol), HOAt (26 mg, 0.19 mmol), triethylamine and 4-Methylamino-piperidine-1-carboxylic acid tert-butyl ester (606 mg, 2.8 mmol). The reaction mixture was stirred for 18 h at Rt. The solvent was removed in vacuo and the yellow residue was redissolved in dichloromethane (80 ml) and was washed with 1M hydrochloric acid (2×80 ml), 1M sodium carbonate (2×80 ml), brine (1×80 ml) and dried over anhydrous magnesium sulphate to yield a clear oil (2 g). Flash chromatography (2% MeOH, dichloromethane) yielded the title compound as a white foam (870 mg, 100%) which contained a slight impurity. HPLC. 6.9 min (85%); LRMS: +ve ion 462 (M+1, 5%), 484 (M+Na, 20%). The compound was progressed to the next step without any further purification.

Step 3

4-[(2-Amino-3,3-dimethyl-butyryl)-methyl-amino]-piperidine-1-carboxylic acid tert-butyl ester

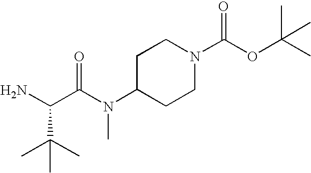

To a solution of 4-[(2-Benzyloxycarbonylamino-3,3-dimethyl-butyryl-methyl-amino]-piperidine-1-carboxylic acid tert-butyl ester (500 mg, 1.1 mmol) in EtOH (10 ml) was added palladium on carbon (50 mg, 10% w/w), hydrogen was bubbled through the reaction mixture for 2 h. The mixture was stirred under a blanket of hydrogen for 18 h at Rt. The Pd/C was filtered off and the solvent removed in vacuo to yield the title compound as a yellow oil which was progressed to the next step without further purification. LRMS: +ve ion 328 (M+1, 100%).

Step 4

4-({2-[3-(Benzyloxy-formyl-amino)-2-cyclopentylmethylpropionylamino]-3,3-dimethyl-butyryl}-methyl-amino)-piperidine-1-carboxylic acid tert-butyl ester

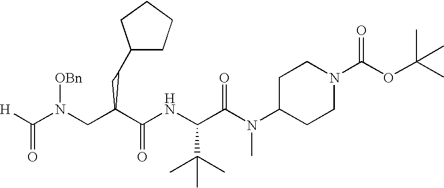

To a solution of of 3-(Benzyloxy-formyl-amino)-2R-cyclopentylmethyl-propionic acid (288 mg, 0.94 mmol) in dichloromethane/DMF, (4:1, 25 ml) was added WSC (198 mg, 1.04 mmol), HOAt (13 mg, 94.5 μmol), triethylamine (196 μl, 1.4 mmol) and 4-[(2-Amino-3,3-dimethyl-butyryl)-methyl-amino]-piperidine-1-carboxylic acid tert-butyl ester (340 mg, 1.04 mmol) at Rt. The reaction mixture was stirred for 18 h at Rt and the solvent was removed in vacuo to yield a yellow oil which was redissolved in dichloromethane (70 ml) and was washed with 1M hydrochloric acid (1×50 ml), 1M sodium carbonate (1×50 ml), brine (1×50 ml) and was dried over anhydrous magnesium sulphate. The solvent was removed under vacuum to yield a clear oil (517 mg). Flash chromatography gradient 2:1 to 1:1 hexane/ethyl acetate yielded the title compound as a white foam (300 mg, 52%). HPLC. 7.1 min (85%); LRMS: +ve ion 462 (M+1, 5%), 484 (M+Na, 20%)

Step 5

2-[3-(Benzyloxy-formyl-amino)-2-cyclopentylmethyl-propionylamino]-3,3,N-trimethyl-N-piperidin-4-yl-butyramide

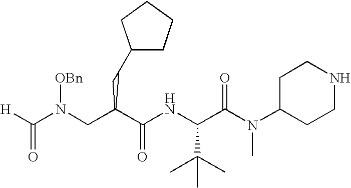

To a solution of 4-({2-[3-(Benzyloxy-formyl-amino)-2-cyclopentylmethyl-propionylamino]-3,3-dimethyl-butyryl}-methyl-amino)-piperidine-1-carboxylic acid tert-butyl ester in dichloromethane (5 ml) was added acetic acid (1 ml) and then boron trifluoride etherate (91 μl, 0.71 mmol) at 0° C. The reaction mixture was stirred at 0 C for 1.25 h, more dichloromthane was added (15 ml) and the organic layer was then washed with 1M sodium carbonate (1×30 ml), dried over anhydrous magnesium sulphate and the solvent was removed in vacuo to yield the title compound as a clear oil (98 mg, 78%). HPLC. 5.2 min (70%), salt formation due to secondary nitrogen is likely hence the purity; LRMS: +ve ion 515 (M+1, 100%). ¹H-NMR (250 MHz), δ (CDCl₃) 8.14 (0.6H, brs, 8.14), 7.87 (0.4H, brs, 7.87), 7.37-7.26 (5H, m), 6.29 (0.4H, d, J 9.4 Hz), 6.25 (0.6H, d, J 9.4 Hz), 5.01-4.71(3H, m), 4.55-4.48 (1H, m), 3.8 (2H, brm), 3.20-3.10 (3H, m), 2.99 (2H, s), 2.81 (1H, s), 2.74-2.63 (2H, m), 2.60-2.55 (1H, m), 1.81-1.37 (13H, m), 1.07-0.97 (2H, m), 0.95-0.92 (11H, m).

Step 6

2-[2-Cyclopentylmethyl-3-(formyl-hydroxy-amino)propionylamino]-3,3,N-trimethyl-N-piperidin-4-yl-butyramide (Intermediate 1)

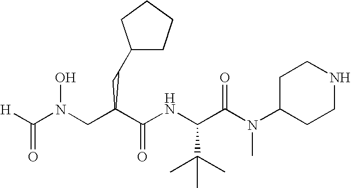

To a solution of 2-[3-(Benzyloxy-formyl-amino)-2-cyclopentylmethyl-propionylamino]-3,3,N-trimethyl-N-piperidin-4-yl-butyramide (135 mg, 0.26 mmol) in EtOH (5 ml) was added Pd/C (14 mg, 15% w/w), hydrogen was bubbled through the suspensioin for 2 h and the reaction was then stirred for 18 h at Rt under a blanket of hydrogen. The catalyst was filtered off and the solvent was removed in vacuo to yield the title compound as a white solid (98 mg, 88%); LRMS: +ve ion 425 (M+1, 100%), −ve ion 423 (M−1, 100%). The NMR was complex due to the presence of rotamers and a zwitterion.

### EXAMPLE 1

### 2-[2-Cyclopentyl-3-(formyl-hydroxy-amino)-propionylamino]-N-[1-(4-methoxy-benzoyl)-piperidin-4-yl]-3,3,N-trimethyl-butyramide

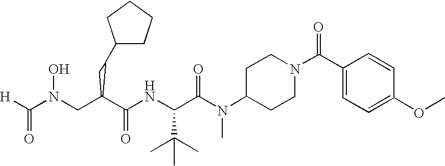

To a solution of 2-[2-Cyclopentylmethyl-3-(formyl-hydroxy-amino)propionylamino]-3,3,N-trimethyl-N-piperidin-4-yl-butyramide (Intermediate 1) (150 mg, 0.4 mmol) in dichloromethane (5 ml) was added 4-methoxy-benzoyl chloride (90.5 mg, 0.5 mmol) and triethylamine (73.4 μl, 0.5 mmol). The reaction mixture was stirred at RT for 18 h. Aminomethyl polystyrene resin (0.89 mmol/g, 3 eq excess) was added and the reaction mixture was left to stir for a further 18 h. The resin was filtered off and the solvent was removed in vacuo, to yield the crude product. The title compound was purified by preparative HPLC to yield a white solid. HPLC: RT: 5.1 min (82% @ 214 nm); ESMS: +ve: 559 (M+1, 20%); −ve: 557 (M−1, 40%).

The minimum inhibitory concentrations of the compound of Example 1 was determined by the protocol described in the Antibacterial Assay section below. The range of MICs determined for the Strep. pneumoniae panel was 0.25-0.5 ngm/litre

Compounds of Examples 2-56 were prepared in a manner analogous to that of Example 1. All compounds were purified by preparative HPLC

| Compound No. | Structure | Analytical Data |
|---|---|---|
| 2 | 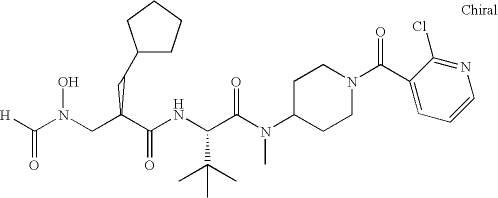 | HPLC: RT: 4.7 min (81% @214 nm) ESMS: +ve: 564 (M + 1, 30%); −ve: 563 (M − 1, 10%) |
| 3 | 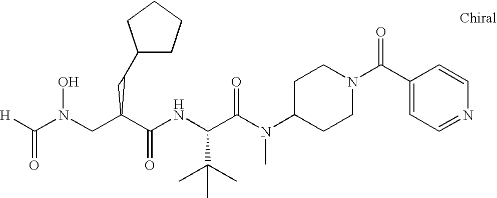 | HPLC: RT: 1.8 min (89% @ 214 nm) ESMS: +ve: 530 (M + 1, 60%), 552 (M + Na, 5%); −ve 528 (M − 1, 10%) |
| 4 | 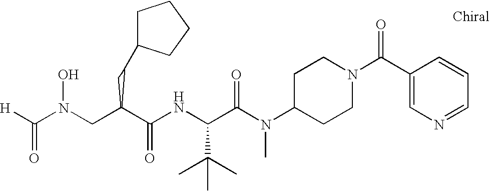 | HPLC: ′RT: 2.0 min (85% @ 214 nm) ESMS: +ve: 530 (M + 1, 100%); −ve: 528 (M − 1, 20%) |
| 5 | 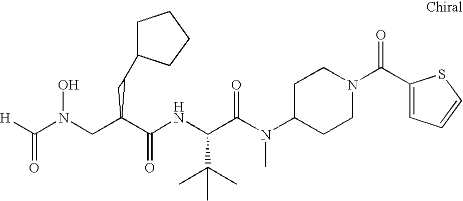 | RT: 5.1 min (80% @ 214 nm) ESMS: +ve: 535 (M + 1, 5%); −ve: 533 (M − 1, 30%) |
| 6 | 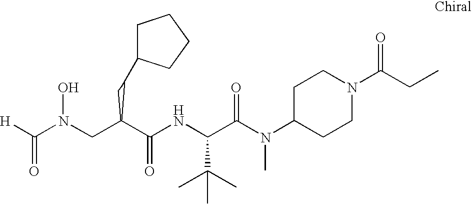 | HPLC: RT: 4.8 min (76%) @ 214 nm ESMS: +ve: 481(M + 1), 503 (M + Na); −ve: 479 (M − 1) |
| 7 | 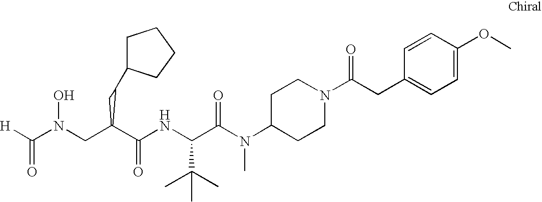 | HPLC: RT: 5.3 min (100%) ESMS: +ve: 573 (M + 1), 595 (M + Na); −ve: 571 (M − 1) |
| 8 | 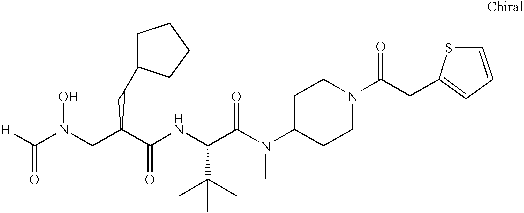 | HPLC: RT: 5.2 min (100%) @214 nm ESMS: +ve: 549 (M + 1), 571 (M + Na); −ve: 547 (M − 1) |
| 9 | 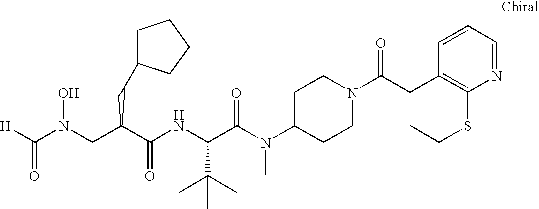 | HPLC: RT: 5.3 min (99%) @ 214 nm ESMS: +ve: 590 (M + 1), 612 (M + Na); −ve: 588 (M − 1) |
| 10 | 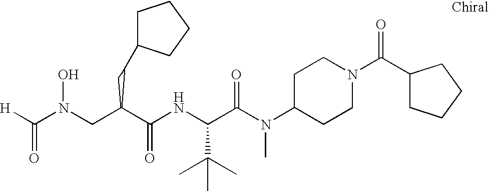 | HPLC: RT: 5.3 min (100%) @214 nm ESMS: +ve: 521 (M + 1), 543 (M + Na); −ve: 519 (M − 1) |
| 11 | 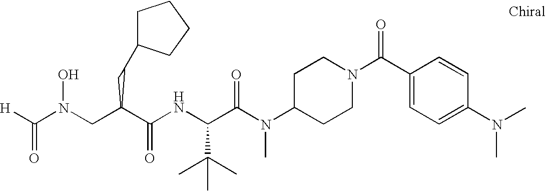 | HPLC: RT: 4.8 min (100%) @214 nm ESMS: +ve: 572 (M + 1), 594 (M + Na) |
| 12 | 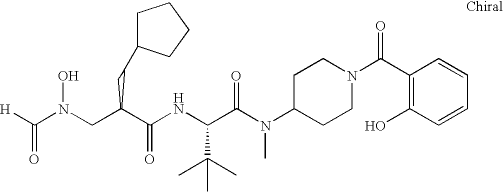 | HPLC: RT: 5.2 min (92%) @ 214 nm ESMS: +ve: 567 (M + Na), 545 (M + 1); −ve: 543 (M − 1) |

| Com- | | ANALYTIC- | |
|---|---|---|---|
| pound | | AL | |
| no | Structure | METHOD | ANALYTICAL DATA |
| 13 | 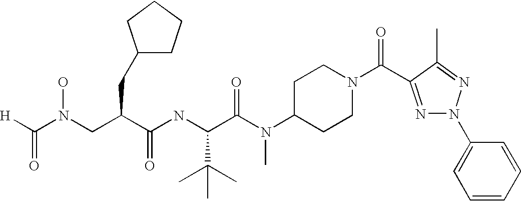 | HPLC ESMS | RT 5.5 min (99% @ 214 nm) +ve: 610 (M + 1, 80%) −ve: 608 (M − 1, 100%) |
| 14 | 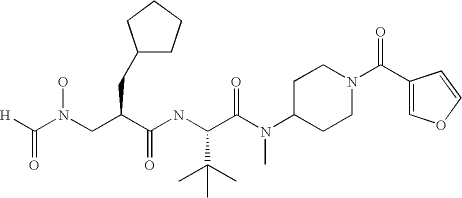 | ESMS HPLC | +ve: 519 (M + 1), 541 (M + Na) −ve: 517 (M − 1) RT: 4.5 min (99%) |
| 15 | 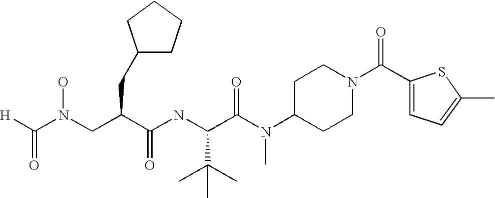 | ESMS HPLC | +ve: 549 (M + 1), 571 (M + Na) −ve: 547 (M − 1) RT: 5.1 min (99%) |
| 16 | 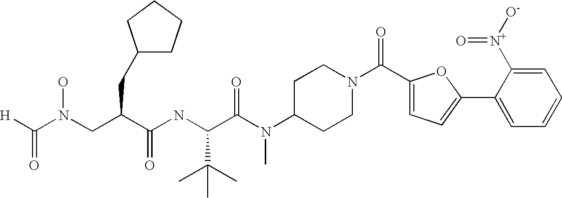 | ESMS HPLC | +ve: 640 (M + 1), 662 (M + Na) −ve: 638 (M − 1) RT: 5.3 min (100%) |
| 17 | 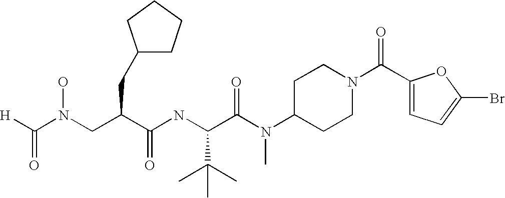 | ESMS HPLC | +ve: 598 (M + 1), 620 (M + Na) −ve: 596 (M − 1) RT: 5.0 min (99%) |
| 18 | 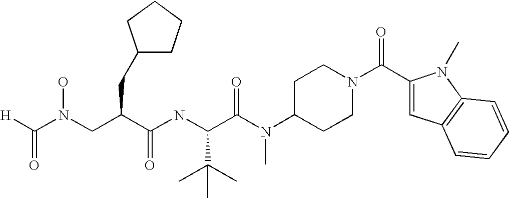 | ESMS HPLC | +ve: 582 (M + a), 604 (M + Na) −ve: 580 (M − 1) RT: 5.4 min (99%) |
| 19 | 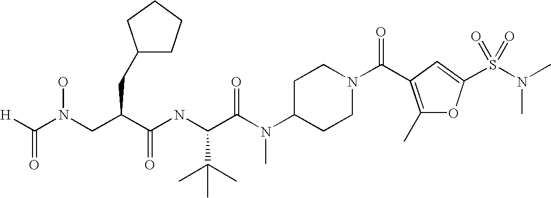 | ESMS HPLC | +ve: 640 (M + 1), 662 (M + Na) −ve: 638 (M − 1) RT: 4.9 (99%) |
| 20 | 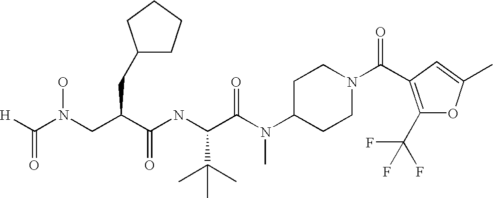 | ESMS HPLC | +ve: 601 (M + 1), 623 (M + Na) −ve: 599 (M − 1) RT: 5.3 min (99%) |
| 21 | 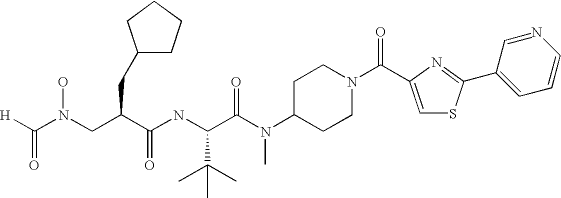 | ESMS HPLC | +ve: 613 (M + 1), 635 (M + Na) −ve: 611 (M − 1) RT: 3.2 min (99%) |
| 22 | 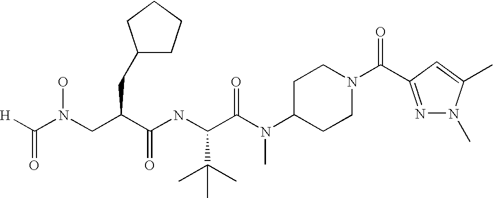 | ESMS HPLC | +ve: 547 (M + 1), 569 (M + Na) −ve: 545 (M − 1) RT: 4.3 min (100%) |
| 23 | 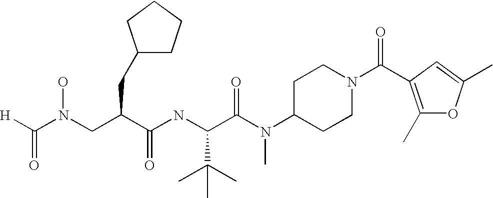 | ESMS HPLC | +ve: 547 (M + 1), 569 (M + Na) −ve: 545 (M − 1) RT: 5.0 min (99%) |
| 24 | 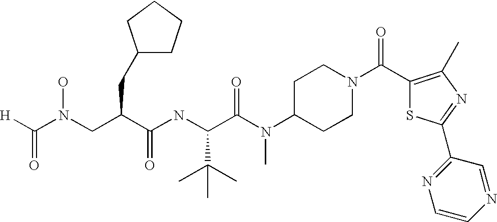 | ESMS HPLC | +ve: 628 (M + 1), 650 (M + Na) −ve: 626 (M − 1) RT: 4.7 min (100%) |
| 25 | 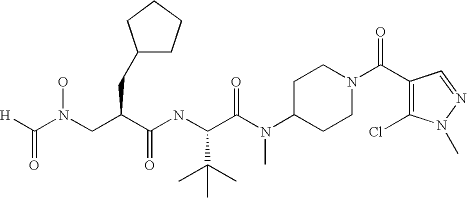 | ESMS HPLC | +ve: 567 (M + 1), 589 (M + Na) −ve: 565 (M − 1) RT: 4.3 min (99%) |
| 26 | 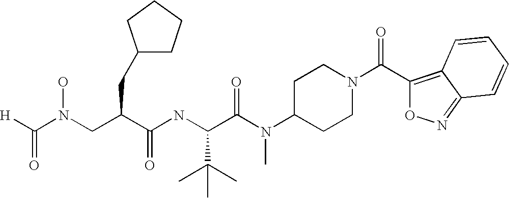 | ESMS HPLC | +ve: 570 (M + 1), 592 (M + Na) −ve: 568 (M − 1) RT: 5.1 min (99%) |
| 27 | 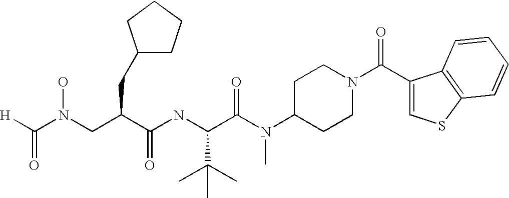 | ESMS HPLC | +ve: 585 (M + 1), 607 (M + Na) −ve: 583 (M − 1) RT: 5.4 min (99%) |
| 28 | 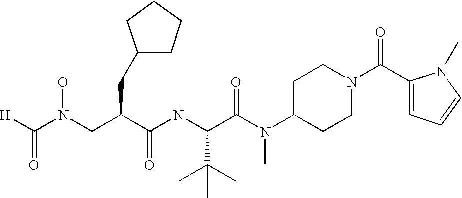 | ESMS HPLC | +ve: 532 (M + 1), 554 (M + Na) −ve: 530 (M − 1) RT: 4.8 min (99%) |
| 29 | 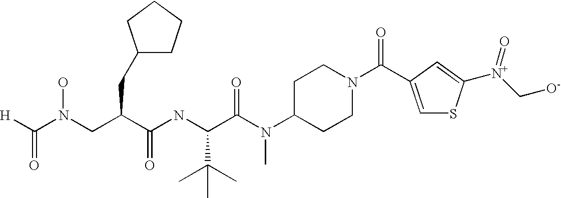 | ESMS HPLC | +ve: 580 (M + 1), 602 (M + Na) −ve: 578 (M − 1) RT: 4.9 min (96% @ 214 nm) |
| 30 | 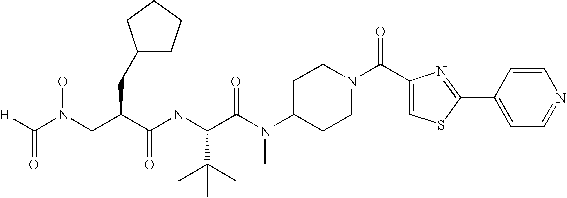 | HPLC ESMS | RT: 1.6 min (100% @ 214 nm) +ve: 613 (M + 1), 635 (M + Na) −ve: 611 (M − 1) |
| 31 | 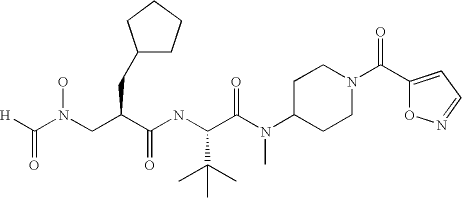 | HPLC ESMS | RT: 4.3 min (99% @ 214 nm) +ve: 520 (M + 1), 542 (M + Na) −ve: 518 (M − 1) |
| 32 | 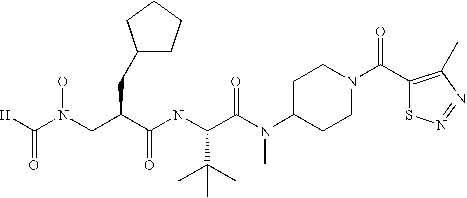 | ESMS HPLC | +ve: 551 (M + 1), 573 (M + Na) −ve: 549 (M − 1) RT: 4.5 min (99%) |
| 33 | 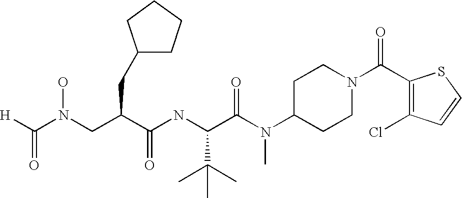 | HPLC ESMS | RT: 5.0 min (98% @ 214 nm) +ve: 569 (M + 1), 591 (M + Na) −ve: 567 (M − 1) |
| 34 | 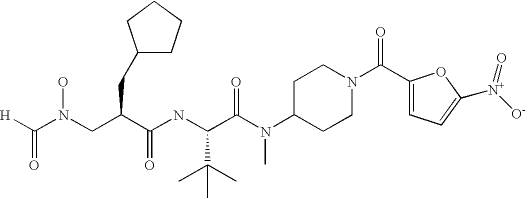 | HPLC ESMS | RT: 4.8 min (90% @ 214 nm) +ve: 564 (M + 1), 586 (M + Na) −ve: 562 (M − 1) |
| 35 | 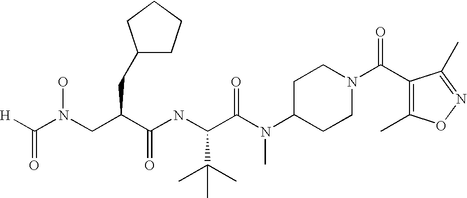 | ESMS HPLC | +ve: 548 (M + 1), 570 (M + Na) −ve: 546 (M − 1) RT: 4.4 min (98% @ 214 nm) |
| 36 | 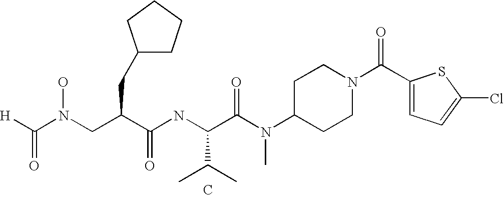 | HPLC ESMS | RT: 5.3 min (99% @ 214 nm) +ve: 569 (M + 1), 591 (M + Na) −ve: 567 (M − 1) |
| 37 | 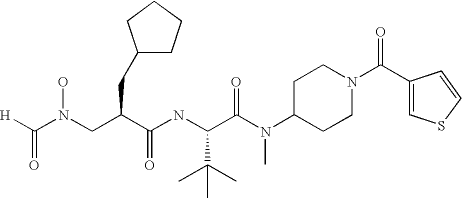 | HPLC ESMS | RT: 4.6 min (99% @ 214 nm) +ve: 640 (M + 1), 662 (M + Na) −ve: 638 (M − 1) |
| 38 | 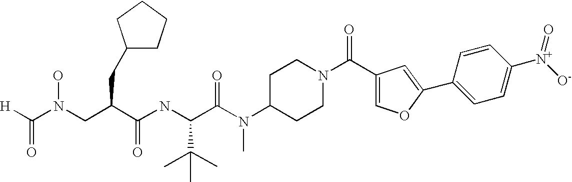 | HPLC ESMS | RT: 5.4 min (97% @ 214 nm) +ve: 640 (M + 1), 662 (M + Na) −ve: 638 (M − 1) |
| 39 | 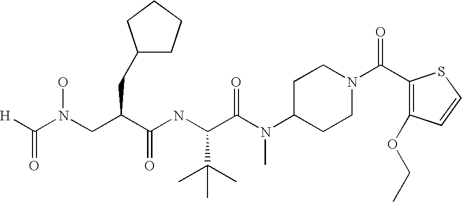 | HPLC ESMS | RT: 5.0 min (99% @ 214 nm) +ve: 579 (M + 1), 601 (M + Na) −ve: 577 (M − 1) |
| 40 | 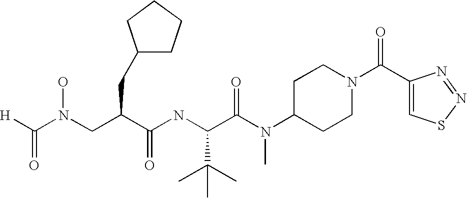 | ESMS HPLC | +ve: 537 (M + 1, 559 (M + Na) −ve: 535 (M − 1) RT: 4.2 min (99% @ 214 nm) |
| 41 | 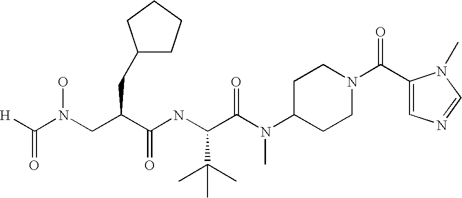 | HPLC ESMS | RT: 5.0 min (96% @ 214 nm) +ve: 533 (M + 1), 555 (M + Na) |
| 42 | 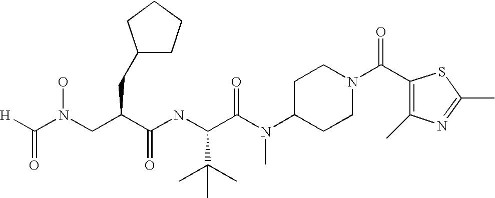 | HPLC ESMS | RT: 4.0 min (98% @ 214 nm) +ve: 564 (M + 1), 586 (M + Na) −ve: 562 (M − 1) |
| 43 | 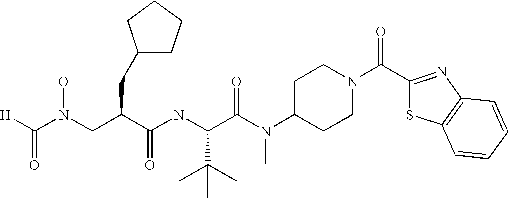 | HPLC ESMS | RT: 5.4 min (>95% @ 214 nm) +ve: 586 (M + 1), 611 (M + Na) −ve: 584 (M − 1) |
| 44 | 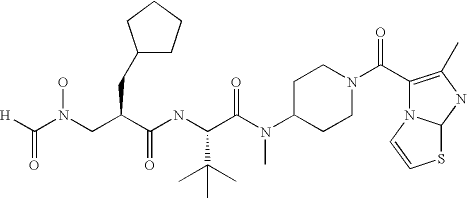 | ESMS HPLC | +ve: 589 (M + 1) RT: 4.5 min (>90%) |
| 45 | 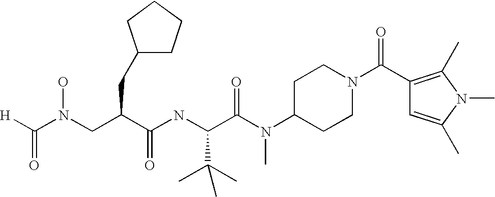 | ESMS HPLC | +ive: 560, 45%, M + 1; 582, 5%, M + Na. 214 nm, 5.20 min, 94%. |
| 46 | 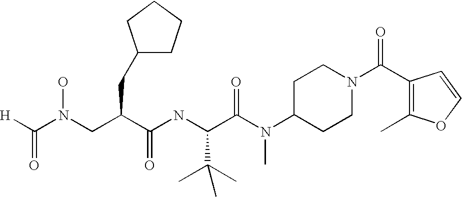 | ESMS HPLC | +ive: 533, 30%, M + 1, 555, 100%, M + Na. 214 nm, 4.84 min, 100% |
| 47 | 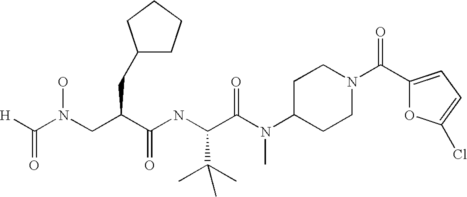 | ESMS HPLC | +ive: 553, 20%, M + 1; 575, 100%, M + Na. 214 nm, 5.14 min, 100% |
| 48 | 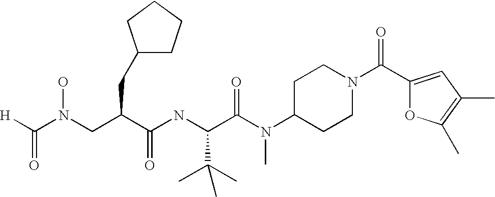 | ESMS HPLC | +ive: 547, 30%, M + 1; 569, 100%, M + Na 214 nm, 5.11 min, 99% |
| 49 | 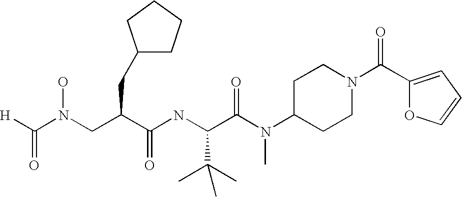 | ESMS HPLC | +ive: 519, 10%, M + 1; 541, 100%, M + Na 214 nm, 4.71 min, 100% |
| 50 | 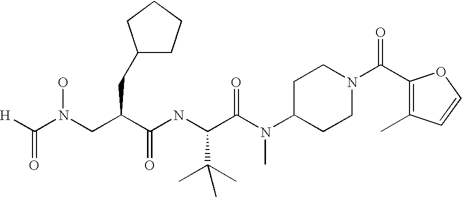 | ESMS HPLC | +ive: 533, 50%, M + 1; 555, 100%, M + Na 214 nm, 4.94 min, 98%. |
| 51 | 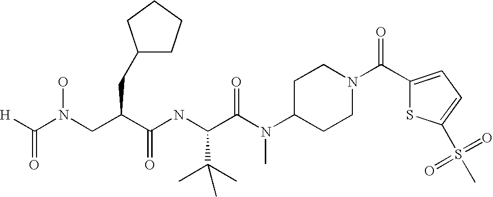 | ESMS HPLC | +ive: 613, 15%, M + 1; 635, 100%, M + Na 214 nm, 4.75 mins, 100% |
| 52 | 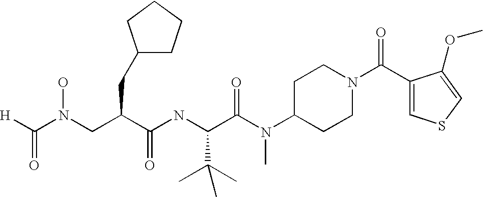 | ESMS HPLC | +ive: 565, 20%, M + 1; 587, 100%, M + Na 214 nm, 4.84 min, 100% |
| 53 | 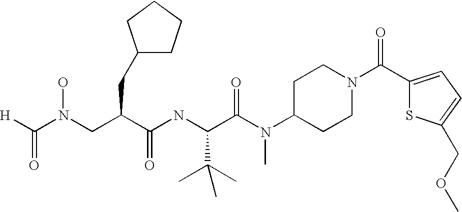 | ESMS HPLC | +ive: 579, 20%, M + 1; 601, 100%, M + Na 214 nm, 4.92 min, 100% |
| 54 | 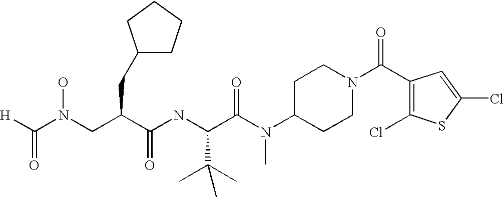 | ESMS HPLC | +ive: 603, 15%, M + 1; 625, 100%, M + Na 214 nm, 5.61 min, 100% |
| 55 | 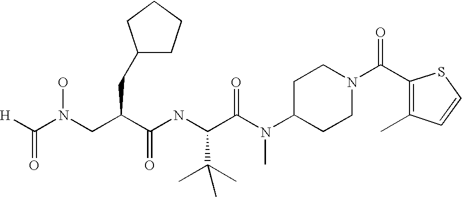 | ESMS HPLC | +ive; 549, 35%, M + 1; 571, 100%, M + Na 214 nm, 5.04 min, 100% |
| 56 | 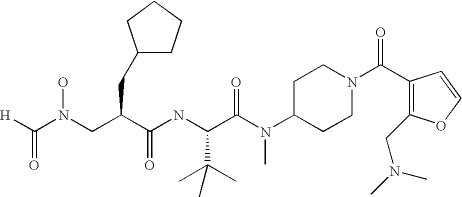 | ESMS HPLC | +ive: 576, 100%, M + 1, 598, 15%, M + Na −ive: 574, 100%, M − 1 214 nm, 4.38 min, 100% |

The minimum inhibitory concentrations of compounds 2 to 56 were also determined by the protocol described in the Antibacterial Assay section below. In each case the range of MICs determined for the Strep. pneumoniae panel is given below, alongside the compound name:
Compound 2: N-[1-(2-Chloro-pyridine-3-carbonyl)-piperidin-4-yl]-2-[2-cyclopentylmethyl-3-(formyl-hydroxy-amino)-propionylamino]-3,3,N-trimethyl-butyramide. MIC 0.25-0.50
Compound 3: 2-[2-Cyclopentylmethyl-3-(formyl-hydroxy-amino)-propionylamino]-3,3,N-trimethyl-N-[1-(pyridine-4-carbonyl)-piperidin4-yl]-butyramide. MIC <0.125-0.25
Compound 4: 2-[2-Cyclopentylmethyl-3-(formyl-hydroxy-amino)-propionylamino]-3,3,N-trimethyl-N-[1-(pyridine-3-carbonyl)-piperidin-4-yl]-butyramide. MIC 1.0-2.0
Compound 5: 2-[2-Cyclopentylmethyl-3-(formyl-hydroxy-amino)-propionylamino]-3,3,N-trimethyl-N-[1-(thiophene-2-carbonyl)-piperidin-4-yl]-butyramide. MIC 0.25-0.50
Compound 6: 2-[2-Cyclopentylmethyl-3-(formyl-hydroxy-amino)-propionylamino]-3,3,N-trimethyl-N-[1-propionyl-piperidin-4-yl]-butyramide. MIC 1.0-2.0
Compound 7: 2-[2-Cyclopentylmethyl-3-(formyl-hydroxy-amino)-propionylamino]-N-{1-[2-(4-methoxy-phenyl)-acetyl]-piperidin-4-yl}-3,3,N-trimethyl-butyramide. MIC 0.25
Compound 8: 2-[2-Cyclopentylmethyl-3-(formyl-hydroxy-amino)-propionylamino]-3,3,N-trimethyl-N-[1-(2-thiophen-2-yl-acetyl)-piperidin-4-yl]-butyramide. MIC 0.25
Compound 9: 2-[2-Cyclopentylmethyl-3-(formyl-hydroxy-amino)-propionylamino]-N-{1-[2-(2-ehtylsulfanyl-pyridin-3-yl)acetyl]-piperidin-4-yl}-3,3,N-trimethyl-butyramide. MIC 0.125-0.25
Compound 10: N-(1-Cyclopentanecarbonyl-piperidin-4-yl)-2[2-cyclopentylmethyl-3-(formyl-hydroxy-amino)-propionylamino]-3,3,N-trimethyl-butyramide. MIC 0.25-0.50
Compound 11: 2-[2-Cyclopentylmethyl-3-(formyl-hydroxy-amino)-propionylamino]-N-[1-(4-dimethylamino-benzoyl)-piperidin-4-yl]-3,3,N-trimethyl-butyramide. MIC <0.125-0.25
Compound 12: 2-[2-Cyclopentylmethyl-3-(formyl-hydroxy-amino)-propionylamino]-N-[1-(2-hydroxy-benzoyl)-piperidin4-yl]-3,3, N-trimethyl-butyramide. MIC <0.125
Compound 13 2S-[2R-Cyclopentylmethyl-3-(formyl-hydroxy-amino)-propionylamino]-3,3,N-trimethyl-N-[1-(5-methyl-2-phenyl-2H-[1,2,3]triazole-4-carbonyl)-piperidin-4-yl]-butyramide. MIC MIC <0.125-0.25
Compound 14 2S-[2R-Cyclopentylmethyl-3-(formyl-hydroxy-amino)-propionylamino]-N-[1-(furan-3-carbonyl)-piperidin-4-yl]-3,3,N-trimethyl-butyramide. MIC <0.125-0.5
Compound 15 2S-[2R-Cyclopentylmethyl-3-(formyl-hydroxy-amino)-propionylamino]-3,3,N-trimethyl-N-[1-(5-methyl-thiophene-2-carbonyl)-piperidin-4-yl]-butyramide MIC <0.125
Compound 16 2S-[2R-Cyclopentylmethyl-3-(formyl-hydroxy-amino)-propionylamino]-3,3,N-trimethyl-N-{1-[5-(2-nitro-phenyl)-furan-2-carbonyl]-piperidin-4-yl}-butyramide MIC <0.125
Compound 17 N-[1-(5-Bromo-furan-2-carbonyl)-piperidin-4-yl]-2S-[2R-cyclopentylmethyl-3-(formyl-hydroxy-amino)-propionylamino]-3,3,N-trimethyl-butyramide MIC <0.125-0.5
Compound 18 2S-[2R-Cyclopentylmethyl-3-(formyl-hydroxy-amino)-propionylamino]-3,3,N-trimethyl-N-[1-(1-methyl-1H-indole-2-carbonyl)-piperidin-4-yl]-butyramide MIC <0.125
Compound 19 2S-[2R-Cyclopentylmethyl-3-(formyl-hydroxy-amino)-propionylamino]-N-[1-(5-dimethylsulfamoyl-2-methyl-furan-3-carbonyl)-piperidin-4-yl]-3,3,N-trimethyl-butyramide MIC <0.125-0.25
Compound 20 2S-[2R-Cyclopentylmethyl-3-(formyl-hydroxy-amino)-propionylamino]-3,3,N-trimethyl-N-[1-(5-methyl-2-trifluoromethyl-furan-3-carbonyl)-piperidin-4-yl]-butyramide MIC <0.125
Compound 21 2S-[2R-Cyclopentylmethyl-3-(formyl-hydroxy-amino)-propionylamino]-3,3,N-trimethyl-N-[1-(2-pyridin-3-yl-thiazole-4-carbonyl)-piperidin-4-yl]-butyramide MIC <0.125
Compound 22 2S-[2R-Cyclopentylmethyl-3-(formyl-hydroxy-amino)-propionylamino]-N-[1-(1,5-dimethyl-1 H-pyrazole-3-carbonyl)-piperidin-4-yl]-3,3,N-trimethyl-butyramide MIC <0.125-0.5
Compound 23 2S-[2R-Cyclopentylmethyl-3-(formyl-hydroxy-amino)-propionylamino]-N-[1-(2,5-dimethyl-furan-3-carbonyl)-piperidin-4-yl]-3,3,N-trimethyl-butyramide MIC <0.125-0.25
Compound 24 2S-[2R-Cyclopentylmethyl-3-(formyl-hydroxy-amino)-propionylamino]-3,3,N-trimethyl-N-[1-(4-methyl-2-pyrazin-2-yl-thiazole-5-carbonyl)-piperidin-4-yl]-butyramide MIC <0.125
Compound 25 N-[1-(5-Chloro-1-methyl-1H-pyrazole-4-carbonyl)-piperidin-4-yl]-2S-[2R-cyclopentylmethyl-3-(formyl-hydroxy-amino)-propionylamino]-3,3,N-trimethyl-butyramide MIC <0.125-0.5
Compound 26 N-[1-(Benzo[c]isoxazole-3-carbonyl)-piperidin-4-yl]-2S-[2R-cyclopentylmethyl-3-(formyl-hydroxy-amino)-propionylamino]-3,3,N-trimethyl-butyramide MIC <0.1250.25
Compound 27 N-[1-(Benzo[b]thiophene-3-carbonyl)-piperidin-4-yl]-2S-[2R-cyclopentylmethyl-3-(formyl-hydroxy-amino)-propionylamino]-3,3,N-trimethyl-butyramide MIC <0.1250.25
Compound 28 2S-[2R-Cyclopentylmethyl-3-(formyl-hydroxy-amino)-propionylamino]-3,3,N-trimethyl-N-[1-(1-methyl-1H-pyrrole-2-carbonyl)-piperidin-4-yl]-butyramide MIC <0.5-1.0
Compound 29 2S-[2R-Cyclopentylmethyl-3-(formyl-hydroxy-amino)-propionylamino]-3,3,N-trimethyl-N-[1-(5-nitro-thiophene-3-carbonyl)-piperidin-4-yl]-butyramide MIC <0.125-0.25
Compound 30 2S-[2R-Cyclopentylmethyl-3-(formyl-hydroxy-amino)-propionylamino]-3,3,N-trimethyl-N-[1-(2-pyridin-4-yl-thiazole-4-carbonyl)-piperidin-4-yl]-butyramide MIC <0.125-0.25
Compound 31 2S-[2R-Cyclopentylmethyl-3-(formyl-hydroxy-amino)-propionylamino]-N-[1-(isoxazole-5-carbonyl)-piperidin-4-yl]-3,3,N-trimethyl-butyramide MIC <0.125-0.5
Compound 32 2S-[2R-Cyclopentylmethyl-3-(formyl-hydroxy-amino)-propionylamino]-3,3,N-trimethyl-N-[1-(4-methyl-[1,2,3]thiadiazole-5-carbonyl)-piperidin-4-yl]-butyramide MIC <0.25-0.5
Compound 33 N-[1-(3-Chloro-thiophene-2-carbonyl)-piperidin-4-yl]-2S-[2R-cyclopentylmethyl-3-(formyl-hydroxy-amino)-propionylamino]-3,3,N-trimethyl-butyramide MIC <0.125-0.5
Compound 34 2S-[2R-Cyclopentylmethyl-3-(formyl-hydroxy-amino)-propionylamino]-3,3,N-trimethyl-N-[1-(5-nitro-furan-2-carbonyly piperidin-4-yl]-butyramide MIC <0.125
Compound 35 2S-[2R-Cyclopentylmethyl-3-(formyl-hydroxy-amino)-propionylamino]-N-[1-(3,5-dimethyl-isoxazole-4-carbonyl)-piperidin-4-yl]-3,3,N-trimethyl-butyramide MIC <0.125
Compound 36 N-[1-(5-Chloro-thiophene-2-carbonyl)-piperidin-4-yl]-2S-[2R-cyclopentylmethyl-3-(formyl-hydroxy-amino)-propionylamino]-3,3,N-trimethyl-butyramide MIC <0.125
Compound 37 2S-[2R-Cyclopentylmethyl-3-(formyl-hydroxy-amino)-propionylamino]-3,3,N-trimethyl-N-[1-(thiophene-3-carbonyl)-piperidin-4-yl]-butyramide MIC <0.125-0.5
Compound 38 2S-[2R-Cyclopentylmethyl-3-(formyl-hydroxy-amino)-propionylamino]-3,3,N-trimethyl-N-{1-[5-(4-nitro-phenyl)-furan-3-carbonyl]-piperidin-4-yl}-butyramide MIC <0.125-0.25
Compound 39 2S-[2R-Cyclopentylmethyl-3-(formyl-hydroxy-amino)-propionylamino]-N-[1-(3-ethoxy-thiophene-2-carbonyl)-piperidin-4-yl]-3,3,N-trimethyl-butyramide MIC <0.125-0.25
Compound 40 2S-[2R-Cyclopentylmethyl-3-(formyl-hydroxy-amino)-propionylamino]-3,3,N-trimethyl-N-[1-([1,2,3]thiadiazole4-carbonyl)-piperidin-4-yl]-butyramide MIC <0.125
Compound 41 2S-[2R-Cyclopentylmethyl-3-(formyl-hydroxy-amino)-propionylamino]-3,3-dimethyl-N-[1-(3-methyl-3H-imidazole-4-carbonyl)-piperidin-4-yl]-N-methylbutyramide MIC <0.125-0.25
Compound 42 2S-[2R-Cyclopentylmethyl-3-(formyl-hydroxy-amino)-propionylamino]-N-[1-(2,4-dimethyl-thiazole-5-carbonyl)-piperidin-4-yl]-3,3,N-trimethyl-butyramide MIC <0.5-1.0
Compound 43 N-[1-(Benzothiazole-2-carbonyl)-piperidin-4-yl]-2S-[2R-cyclopentylmethyl-3-(formyl-hydroxy-amino)-propionylamino]-3,3,N-trimethyl-butyramide MIC <0.125-0.25
Compound 44 2S-[2R-Cyclopentylmethyl-3-(formyl-hydroxy-amino)-propionylamino]-3,3,N-trimethyl-N-[1-(6-methyl-7,7a-dihydro-imidazo[2,1-b]thiazole-5-carbonyl)-piperidin-4-yl]-butyramide MIC 1.0-4.0
Compound 45 2S-[2R-Cyclopentylmethyl-3-(formyl-hydroxy-amino)-propionylamino]-3,3,N-trimethyl-N-[1-(1,2,5-trimethyl-1H-pyrrole-3-carbonyl)-piperidin-4-yl]-butyramide MIC 4.0-8.0
Compound 46 2S-[2R-Cyclopentylmethyl-3-(formyl-hydroxy-amino)-propionylamino]-3,3-dimethyl-N-[1-(2-methyl-furan-3-carbonyl)-piperidin-4-yl]-N-methylbutyramide MIC 0.06-0.5
Compoun 47 N-[1-(5-Chloro-furan-2-carbonyl)-piperidin-4-yl]-2S-[2R-Cyclopentylmethyl-3-(formyl-hydroxy-amino)-propionylamino]-3,3,N-trimethyl-butyramide MIC 0.06-0.5
Compound 48 2S-[2R-Cyclopentylmethyl-3-(formyl-hydroxy-amino)-propionylamino]-N-[1-(4,5-dimethyl-furan-2-carbonyl)piperidin-4-yl]-3,3,N-trimethyl-butyramide MIC 0.06-0.5
Compound 49 2S-[2R-Cyclopentylmethyl-3-(formyl-hydroxy-amino)-propionylamino]-N-[1-(furan-2-carbonyl)-piperidin-4-yl]-3,3,N-trimethyl-butyramide MIC 0.125-1.0
Compound 50 2S-[2R-Cyclopentylmethyl-3-(formyl-hydroxy-amino)-propionylamino]-3,3-dimethyl-N-[1-(3-methyl-furan-2-carbonyl)-piperidin-4-yl]-N-methylbutyramide MIC 0.06-1.0
Compound 51 2S-[2R-Cyclopentylmethyl-3-(formyl-hydroxy-amino)-propionylamino]-N-[1-(5-methanesulphonyl-thiophene-2-carbonyl)-piperidin-4-yl]-3,3,N-trimethyl-butyramide MIC 0.125-0.5
Compound 52 2S-[2R-Cyclopentylmethyl-3-(formyl-hydroxy-amino)-propionylamino]-N-[1-(4-methoxyl-thiophene-3-carbonylypiperidin-4-yl]-3,3,N-trimethyl-butyramide MIC 0.125-0.25
Compound 53 2S-[2R-Cyclopentylmethyl-3-(formyl-hydroxy-amino)-propionylamino]-N-[1-(5-methoxymethyl-thiophene-2-carbonyl)-piperidin-4-yl]-3,3,N-trimethyl-butyramide MIC 0.125-0.5
Compound 54 2S-[2R-Cyclopentylmethyl-3-(formyl-hydroxy-amino)-propionylamino]-N-[1-(2,5-dichloro-thiophene-2-carbonyl)-piperidin-4-yl]-3,3,N-trimethyl-butyramide MIC <0.03-0.5
Compound 55 2S-[2R-Cyclopentylmethyl-3-(formyl-hydroxy-amino)-propionylamino]-3,3-dimethyl-N-[1-(3-methyl-thiophene-2-carbonyl)-piperidin-4-yl]-N-methylbutyramide MIC 0.125-0.5
Compound 56 2S-[2R-Cyclopentylmethyl-3-(formyl-hydroxy-amino)-propionylamino]-N-[1-(2-dimethylaminomethyl-furan-3-carbonyl)-piperidin-4-yl]-3,3,N-trimethyl-butyramide MIC 0.25-1.0

Antibacterial Assay

Minimal Inhibitory concentrations (MIC) of inhibitors against clinical isolates of S. pneumoniae, obtained from the Public Health and Clinical Microbiology Laboratory, Addenbrooke's Hospital, Hills Road, Cambridge CB2 2QW, UK, were determined by a standard agar plate dilution method following recommendations in British Society for Antimicrobial Chemotherapy Working Party. 1991. “A guide to sensitivity testing British Society for Antimicrobial Chemotherapy, London, United Kingdom”. Briefly Iso-Sensitest agar (pH 7.2: Oxoid, United Kingdom) was employed supplemented with 5% horse blood (Oxoid) and 20 μg of NAD (Sigma) per ml to support growth of fastidious bacteria. The inoculum used was approximately 10⁴ colony forming units of each isolate contained in a volume of 1 μl. Plates were incubated 18 to 24 hr in air, or for fastidious bacteria an atmosphere enriched with 4-6% carbon dioxide at 35° C. The MIC was determined as the lowest concentration of an antimicrobial tested that inhibited growth of the inoculum, disregarding a single persisting colony or faint haze caused by the inoculation. MICs were expressed as ngm/litre.

1. A compound of formula (II), or a pharmaceutical or veterinarily acceptable salt, hydrate or solvate thereof 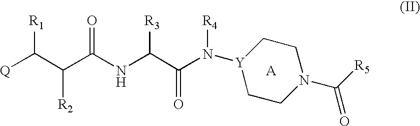
wherein
Q represents a radical of formula —N(OH)CH(═O) or formula —C(═O)NH(OH);
R₁ represents hydrogen, methyl or trifluoromethyl, or, except when Z is a radical of formula —N(OH)CH(═O), a hydroxy, halo or amino group;
R₂ represents a group R₁₀-(V)ₙ-(ALK)ₘ- wherein
R₁₀ represents hydrogen, or a C₁-C₆ alkyl, C₂-C₆ alkenyl, C₂-C₆ alkynyl, cycloalkyl, aryl, or heterocyclyl group, any of which may be unsubstituted or substituted by (C₁-C₆)alkyl, (C₁-C₆)alkoxy, hydroxy, mercapto, (C₁-C₆)alkylthio, amino, halo, trifluoromethyl, cyano, nitro, oxo, —COOH, —CONH₂, —COOR¹, —NHCOR^{A}, —CONHR^{A}, —NHR^{A}, —NR^{A}R^{B}, or —CONR^{A}R^{B} wherein R^{A} and R^{B} are independently a (C₁-C₆)alkyl group and
ALK represents a straight or branched divalent C₁-C₆ alkylene, C₂-C₆ alkenylene, or C₂-C₆ alkynylene radical, and may be interrupted by one or more non-adjacent —NH—, —O— or —S-linkages,
V represents —NH—, —O— or —S—, and
m and n are independently 0 or 1;
R₃ represents the side chain of a natural or non-natural alpha amino acid;
R₄ represents hydrogen or C₁-C₃ alkyl;
Y represents N or CH;
ring A is optionally substituted on one or more ring carbon atoms by C₁-C₃ alkyl, C₁-C₃ alkoxy, or halo; and
R₅ represents a group (IIA), 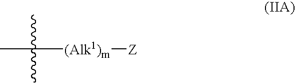
wherein
m is 0 or 1;
Alk¹ represents a divalent C₁-C₃ alkylene radical;
Z represents hydrogen or cycloalkyl, phenyl or heterocyclic which is optionally substituted by
(C₁-C₆)alkyl,
phenyl,
monocyclic 5 or 6-membered heterocyclic,
benzyl,
phenoxy, or (C₁-C₆)alkoxy,
phenylthio or (C₁-C₆)alkylthio, any of which is in turn optionally substituted by:
hydroxy or mercapto,
trifluoromethyl,
oxo,
nitro,
cyano (—CN),
bromo, chloro, fluoro, or iodo,
—COOH, or —COOR^{A},
—CONH₂, —CONHR^{A}, or —CONR^{A}R^{B}
—COR^{A}, —SO₂R^{A},
—NHCOR^{A},
—NH₂, —NHR^{A}, or —NR^{A}R^{B},
wherein R^{A} and R^{B} are independently a (C₁-C₆)alkyl group, or R^{A} and R^{B} taken together with the nitrogen atom to which they are attached form a 5- or 6-membered heterocyclic ring which may be substituted (C₁-C3)alkyl, hydroxy, or hydroxy(C₁-C₃)alkyl.

2. A compound as claimed in claim 1 wherein Z represents cycloalkyl, phenyl or monocyclic-heterocyclic, which is optionally substituted by
(C₁C₆)alkyl, (C₂-C₆)alkenyl, or (C₂-C₆)alkynyl,
phenyl, or halophenyl,
trifluoromethyl,
monocyclic 5 or 6-membered hetrocyclic,
benzyl, or halophenylmethyl,
hydroxy, phenoxy, (C₁-C₆)alkoxy, or hydroxy(C₁-C₆)alkyl,
mercapto, (C₁-C₆)alkylthio or mercapto(C₁-C₆)alkyl,
oxo,
nitro,
cyano (—CN),
bromo, chloro, fluoro, or iodo,
—COOH, or —COOR^{A},
—CONH₂, —CONHR^{A}, or—CONR^{A}R^{B}
—COR^{A}, —SO₂R^{A},
—NHCOR^{A},
—NH₂, —NHR^{A}, or —NR^{A}R^{B},
wherein R^{A} and R^{B} are independently a (C₁-C₆)alkyl group, or R^{A} and R^{B} taken together with the nitrogen atom to which they are attached form a 5- or 6-membered heterocyclic ring which may be substituted by (C₁-C₃)alkyl hydroxy, or hydroxy(C₁-C₃)alkyl.

3. A compound as claimed in claim 1 wherein R₁ is hydrogen.

4. A compound as claimed in claim 1 wherein R₂ is (C₁-C₆)alkyl-, cycloalkyl(C₁-C₆)alkyl-, (C₁-C₃)alkyl-S-(C₁-C₃)alkyl-, or (C₁-C₃)alkyl-O-(C₁-C₃)alkyl-.

5. A compound as claimed in claim 1 wherein R₂ is n-propyl, n-butyl, n-pentyl, cyclopentylmethyl, cyclopentylethyl, cyclohexylmethyl or cyclohexylethyl.

6. A compound as claimed in claim 1
wherein R₃ is
the characterising group of a natural α amino acid, or 4-methoxyphenylmethyl, in which any functional group may be protected, any amino group may be acylated and any carboxyl group present may be amidated; or
a group -[Alk]ₙR₉ where Alk is a (C₁-C₆)alkylene or (C₂-C₆) alkenylene group optionally interrupted by one or more —O—, or —S— atoms or —N(R₁₂)— groups [where R₁₂ is a hydrogen atom or a (C₁-C₆)alkyl group], n is 0 or 1, and R₉ is hydrogen or an optionally substituted phenyl, aryl, heterocyclyl, cycloalkyl or cycloalkenyl group or (only when n is 1) R₉ may additionally be hydroxy, mercapto, (C₁-C₆)alkylthio, amino, halo, trifluoromethyl, nitro, —COOH, —CONH₂, —COOR^{A}, NHCOR^{A}, —CONHR^{A}, —NHR^{A}, —NR^{A}R^{B}, or —CONR^{A}R^{B} wherein R^{A} and R^{B} are independently a (C₁-C₆)alkyl group; or
a benzyl group substituted in the phenyl ring by a group of formula —OCH₂COR₈ where R₈ is hydroxyl, amino, (C₁-C₆)alkoxy, phenyl(C₁-C₆)alkoxy, (C₁-C₆)alkylamino, di((C₁-C₆)alkyl)amino, phenyl(C₁-C₆)alkylamino; or
a heterocyclic (C₁-C₆)alkyl group, either being unsubstituted or mono- or disubstituted in the heterocyclic ring with halo, nitro, carboxy, (C₁-C₆)alkoxy, cyano, (C₁-C₆)alkanoyl, trifluoromethyl(C₁-C₆)alkyl, hydroxy, formyl, amino, (C₁-C₆)alkylaamino, di-(C₁-C₆)alkylamino, mercapto, (C₁-C₆)alkylthio, hydroxy(C₁-C₆)alkyl, mercapto(C₁-C₆)alkyl or (C₁-C₆)alkylphenylmethyl; or
a group-CRₐR_{b}R_{c} in which:
each of Rₐ, R_{b} and R_{c} is independently hydrogen, (C₁-C₆)alkyl, (C₂-C₆)alkenyl, (C₂-C₆)alkynyl, phenyl(C₁-C₆)alkyl, (C₃-C₈)cycloalkyl; or
R_{c} is hydrogen and Rₐ and R_{b} are independently phenyl or heteroaryl; or
R_{c} is hydrogen, (C₁-C₆)alkyl, (C₂-C₆)alkenyl, (C₂-C₆)alkynyl, phenyl(C₁-C₆)alkyl, or (C₃-C₈)cycloalkyl, and Rₐ and R_{b} together with the carbon atom to which they are attached form a 3 to 8 membered cycloalkyl or a 5-to 6-membered heterocyclic ring; or
Rₐ, R_{b} and R_{c} together with the carbon atom to which they are attached form a tricyclic ring; or
Rₐ and R_{b} are each independently (C₁-C₆)alkyl, (C₂-C₆)alkenyl, (C₂-C₆)alkynyl, phenyl(C₁-C₆)alkyl, or a group as defined for R_{c} below other than hydrogen, or Rₐ and R_{b} together with the carbon atom to which they are attached form a cycloalkyl or heterocyclic ring, and R is hydrogen, —OH, —SH, halogen, —CN, —CO₂H, (C₁-C₄) perfluoroalkyl, —CH₂OH, —CO₂(C₁-C₆)alkyl, —O(C₁-C₆)alkyl, —O(C₂-C₆)alkenyl, —S(C₁-C₆)alkyl, —SO(C₁-C₆)alkyl, —SO₂(C₁-C₆)alkyl, —S(C₂-C₆)alkenyl, —SO(C₂-C₆)alkenyl, —SO₂(C₂-C₆)alkenyl or a group -Q-W wherein Q represents a bond or —O—, —S—, —SO— or —SO₂— and W represents a phenyl, phenylalkyl, (C₃-C₈) cycloalkyl, (C₃-C₈) cycloalkylalkyl, (C₄-C₈) cycloalkenyl, (C₄-C₈) cycloalkenylalkyl, heteroaryl or heteroarylalkyl group, which group W may optionally be substituted by one or more substituents independently selected from, hydroxyl, halogen, —CN, —CO₂H, —CO₂(C₁-C₆)alkyl, —CONH₂, —CONH(C₁-C₆)alkyl alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl.

7. A compound as claimed in claim 1 wherein R₃ is methyl, ethyl, n-propyl, n-butyl, benzyl, 4-chlorobenzyl, 4-hydroxybenzyl, phenyl, cyclohexyl, cyclohexylmethyl, pyridin-3-ylmethyl, tert-butoxymethyl, naphthylmethyl, iso-butyl, sec-butyl, tert-butyl, 1-benzylthio-1-methylethyl, 1-methylthio-1-methylethyl, 1-mercapto-1-methylethyl, 1-methoxy-1-methylethyl, 1-hydroxy-1-methylethyl, 1-fluoro-1-methylethyl,hydroxymethyl, 2-hydroxethyl, 2-carboxyethyl, 2-methylcarbamoylethyl, 2-carbamoylethyl, or 4-aminobutyl.

8. A compound as claimed in claim 1 wherein R₃ is tert-butyl, isobutyl, benzyl, isopropyl or methyl.

9. A compound as claimed in claim 1 wherein R₄ is methyl.

10. A compound, as claimed in claim 1 wherein in the group R₅, m is 1, and Alk¹ is —(CH₂)— or —(CH₂CH₂)—.

11. A compound as claimed in claim 1 wherein, in the group R₅, Z is a phenyl, pyridyl, thienyl, furanyl, pyranyl, pyrolyl, diazolyl, triazolyl, thiazolyl, thiadiazolyl, oxazolyl, ozadiazolyl, indolyl, benzisozazolyl, benzthiazolyl or imidazothiazolyl ring, optionally substituted as specified in claim 1.

12. (canceled)

13. A compound as claimed in claim 1 wherein the compound is one specifically named and/or exemplified herein, or is the hydroxamate (Q represents a radical of formula —C(═O)NH(OH)) analogue thereof.

14. A method for the treatment of bacterial infections in humans and non-human mammals, which comprises administering to a subject suffering such infection an antibacterially effective dose of a compound as claimed in claim 1.

15. A method for the treatment of bacterial contamination by applying an antibacterially effective amount of a compound as claimed in claim 1 to the site of contamination.

16. The use of a compound as claimed in claim 1 in the manufacture of an antibacterial composition.

17. A pharmaceutical or veterinary composition comprising a compound as claimed in claim 1 together with a pharmaceutical of veterinarily acceptable carrier.

18. A compound as claimed in claim 1 wherein, in the group R₅, Z is a cyclopentyl, cyclohexyl, phenyl, morpholinyl, pyrimidin-2-yl, 1,2,3-thiadiazol-5-yl, 1,4-thiazol-5-yl, benzofuran-2-yl, 2- or 3-furanyl, 2- or 3-thienyl, 2- or 3-pyranyl, 2-, 3- or 4-pyrrolyl, 3-, 4-or 5-pyrazolyl, 3-, 4- or 5-isoxazolyl, or 2-, 3-or 4-pyridyl ring any of which may optionally be substituted by hydroxy, methoxy, ethoxy, mercapto, methylthio, ethylthio, methyl, ethyl, trfluoromethyl, fluoro, chloro, amino, methylamino, or dimethylamino.
